(19) United States
(12) Patent Application Publication
(10) Pub. No.: US 2025/0270230 A1
(43) Pub. Date: Aug. 28, 2025

(54) NOVEL SYNTHETIC STEPS FOR THE MANUFACTURING METHOD OF THE PDE4B-INHIBITOR 1-({(5R)-2-[4-(5-CHLOROPYRIMIDINE-2-YL)PIPERIDINE-1-YL]-5-OXIDO-6,7-DIHYDROTHIENO[3,2-D]PYRIMIDINE-4-YL}AMINO)CYCLOBUTYL]METHANOL

(71) Applicant: Boehringer Ingelheim International GmbH, Ingelheim am Rhein (DE)

(72) Inventors: Jocelyn M. ABELLA, Danbury, CT (US); Tobias BRODMANN, Ingelheim (DE); Dabing CHEN, Southbury, CT (US); Mai Thi Quynh DANG, Ingelheim (DE); Rogelio P. FRUTOS, Sandy Hook, CT (US); Joe Ju GAO, Southbury, CT (US); Frank GERSTMANN, Frankfurt am Main (DE); Robert HAGENKOETTER, Ingelheim (DE); Ruoshi LI, Ridgefield, CT (US); Daniel Jens MARCKART, Budenheim (DE); Jason Alan MULDER, New Milford, CT (US); Jana ORBEN, Gutenberg (DE); Christian STANGE, Ingelheim am Rhein (DE); Thomas G. TAMPONE, Southbury, CT (US); Dirk WEBER, Mainz (DE)

(21) Appl. No.: 18/983,522

(22) Filed: Dec. 17, 2024

(30) Foreign Application Priority Data
Dec. 19, 2023 (EP) 23217894.7

(51) Int. Cl. C07D 495/04; C07D 401/04

(52) U.S. Cl. CPC C07D 495/04; C07D 401/04

(57) ABSTRACT

The invention is directed to an improved method of manufacturing the PDE4B-inhibitor of formula XX

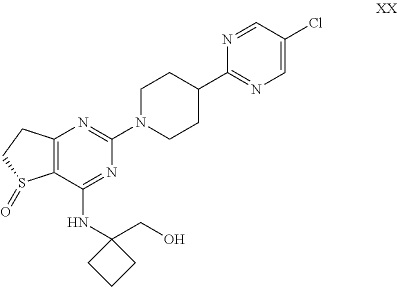 [SMILES: O=[S@@]1CCc2nc(N3CCC(c4ncc(Cl)cn4)CC3)nc(NC3(CO)CCC3)c21]

The phosphodiesterase 4-inhibitor (PDE4-inhibitor) of formula XX

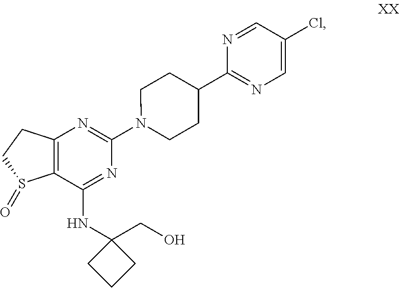 [SMILES: O=[S@@]1CCc2nc(N3CCC(c4ncc(Cl)cn4)CC3)nc(NC3(CO)CCC3)c21]

which preferentially inhibits the PDE4B subunit as well as a method of manufacture of this PDE4B-inhibitor of formula XX has been disclosed in U.S. Pat. No. 8,609,670.

As shown by Phase II clinical data the PDE4B-inhibitor of formula XX showed a reduction in the rate of lung function decline in patients with idiopathic pulmonary fibrosis (IPF) within 12 weeks of treatment. This reduction in lung function decline could be observed in both, in IPF patients who were not on approved antifibrotics as background treatment and in IPF patients who were already taking approved antifibrotics as background treatment such as either Nintedanib or Pirfenidon (https://www.boehringer-ingelheim.us/press-release/boehringer-ingelheims-latest-investigational-treatment-slowed-lung-function-decline).

### 1 BACKGROUND OF THE INVENTION AND ADVANTAGES OF THE IMPROVED PROCESS STEPS

In U.S. Pat. No. 8,609,670 the synthesis of the PDE4B-inhibitor of formula XX in laboratory scale is disclosed. The method of synthesis of the PDE4B-inhibitor of formula XX includes the synthesis of the intermediate G, which is intermediate 5-chloro-2-(4-piperidinyl)-pyrimidine hydrochloride and is formed by converting 4-cyanopiperidine in a two-step sequence with (Z)—N-(2-chloro-3-(dimethylamino)allylidene)-N-methylmethan-aminium hexafluorophosphate:

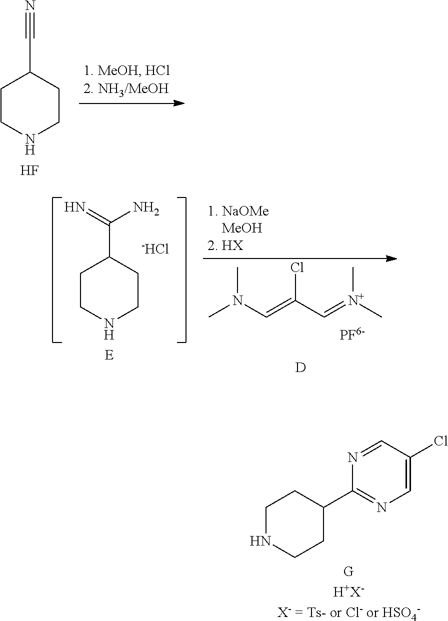

Further, U.S. Pat. No. 8,609,670 discloses the synthesis of the intermediates 1-[(2-chloro-6,7-dihydrothieno[3,2-d]pyrimidin-4-yl)amino]-cyclobutanemethanol and 1-[[(5R)-2-chloro-6,7-dihydro-5-oxidothieno[3,2-d]pyrimidin-4-yl]amino]-cyclobutanemethanol.

U.S. Pat. No. 8,471,010 discloses the synthesis of dihydrothieno[3,2-d]pyrimidine diols. The chlorination of the corresponding diole yielding 2,4-dichloro-thieno-pyrimidine (XIV) is described in U.S. Pat. No. 10,745,411. Herein, for the chlorination step, 0.68 eq. phosphorus oxychloride (POCI3) have been dosed at a temperature of 105-110° C. Then, at an internal temperature of 110° C., another 1.32 eq. POCl₃ were added.

In K.-T.-Wong et al, Org. Lett. 2002, 4 (4), 513 a palladium-catalyzed Suzuki-coupling using arylboronates and 5-bromo-2-iodo-pyrimidine as coupling partner is described. However, the expansion to 5-chloro-2-iodo-pyrimidine (IV) has not been described in Wong et al. For the cross-coupling, 1.0 mol % Pd(PPh₃)₄ has been used.

In P. R. Eastwood, Tetrahedron Lett., 2000, 3705 a palladium-mediated cross-coupling using aryl-bromides, -iodides or -triflates and cyclic N-protected vinyl boronates as coupling partners is described. Hereby, carboxybenzyl (Cbz) is used as N-protecting group. However, Eastwood et al does not disclose that chlorinated aryl-substrates are also tolerated in course of the cross-coupling. For the cross-coupling, 6.0 mol % PdCl₂dppf has been used as a catalyst.

The closest reference should be U.S. Pat. No. 8,609,670B, wherein a manufacturing method of Example 2 (which is identical to the PDE4B-inhibitor of formula XX according to the invention) is disclosed.

However, the method of manufacturing Example 2 of U.S. Pat. No. 8,609,670B has many disadvantages, in particular for large scale manufacturing of the PDE4B-inhibitor of formula XX.

Many of these disadvantages in the method of manufacturing Example 2 according to U.S. Pat. No. 8,609,670B have been significantly improved in the method of manufacturing the PDE4B-inhibitor of formula XX

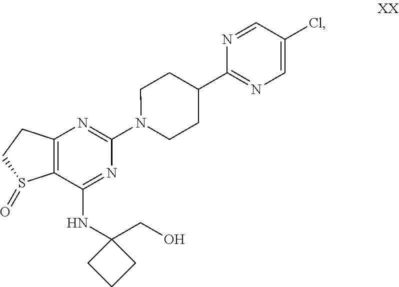 [SMILES: O=[S@@]1CCc2nc(N3CCC(c4ncc(Cl)cn4)CC3)nc(NC3(CO)CCC3)c21]

according to Scheme 1 of the instant invention:

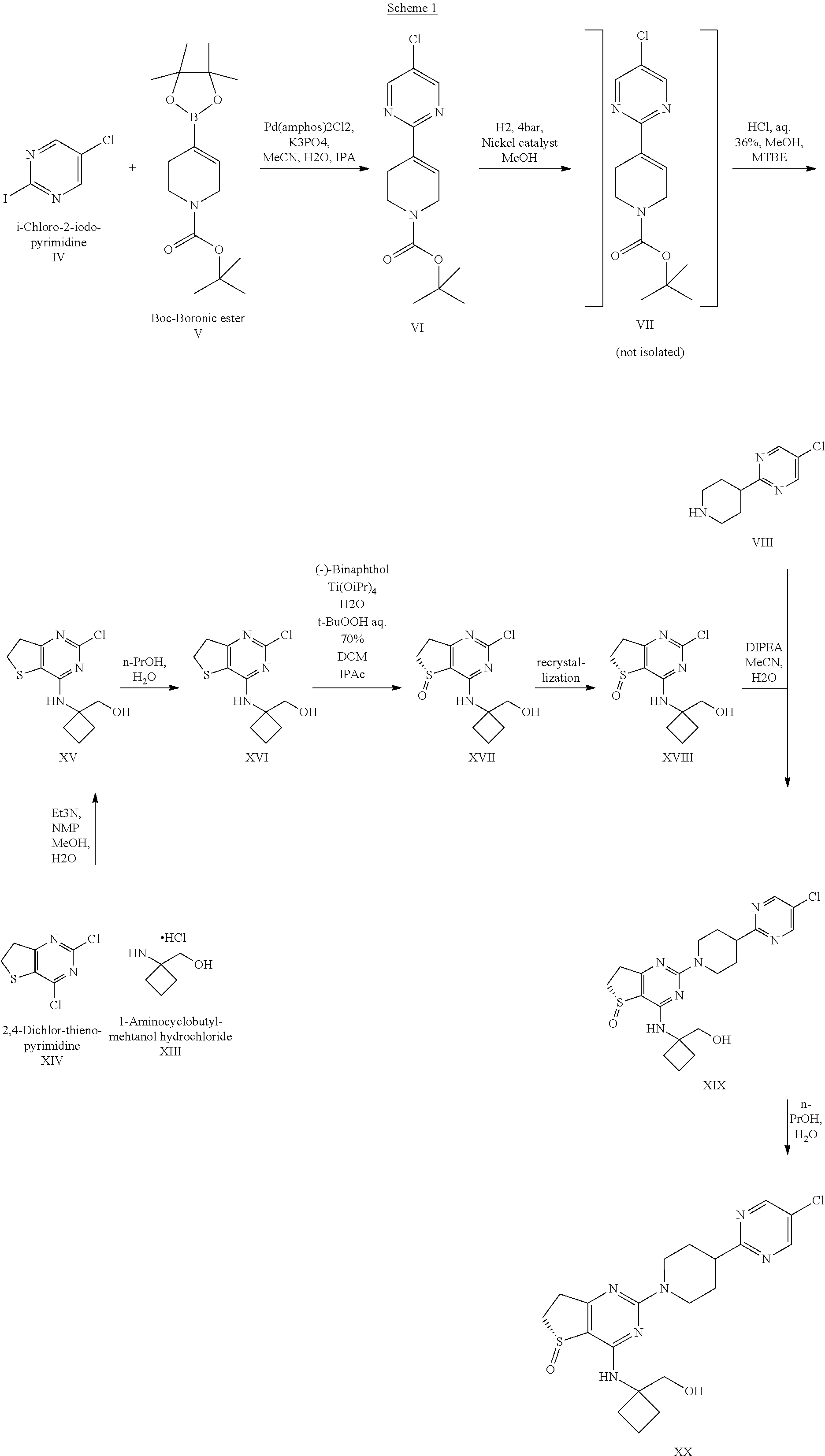

Compared to the method of manufacturing method of Example 2 as described in U.S. Pat. No. 8,609,670B the method of manufacture of the PDE4-inhibitor of formula XX according to scheme 1 of the instant invention includes several new and amended synthesis steps including:
1. the alternative and amended synthesis of Intermediate VIII via Suzuki-reaction type (better control of impurities by avoiding the formation of N-methylation at the piperidine ring and isolation of Intermediate VIII with a higher assay than described in U.S. Pat. No. 8,609,670B, better process economy by lower amounts of the catalyst Pd (amphos)₂Cl₂ than described in a) K.-T.-Wong et al, Org. Lett. 2002, 4 (4), 513; b) P. R. Eastwood, Tetrahedron Lett., 2000, 3705)
2. an amended synthesis of Intermediate XV with higher process economy and higher yields than the ones described in U.S. Pat. No. 8,609,670B
3. an additional crystallization step for Intermediate XV (in comparison to the process as described in U.S. Pat. No. 8,609,670B) to yield a triethylammonium-salt-free Intermediate XVI, which is important to yield an enantiomerically pure Intermediate XVII in the downstream step
4. More cost-efficient stereoselective oxidization step to yield Intermediate XVII by a new preincubation step for S-(−)-binaphthol in dichloromethane and the catalyst Ti(OiPr)₄
5. an additional recrystallization step of Intermediate XVII after the stereoselective oxidation step (in comparison to the process as described in U.S. Pat. No. 8,609,670B) to yield an Intermediate XVIII with a titanium-content of below 30 ppm
6. a more cost-efficient and more environment-friendly synthesis of Intermediate XIX (in comparison to the process as described in U.S. Pat. No. 8,609,670B)
7. an improved final recrystallization process of Compound of formula XX with an improved conversion rate from anhydrous crystalline Form A to anhydrous crystalline Form B (in comparison to the process as described in U.S. Pat. No. 8,609,670B)

### 1.1 Manufacturing of Intermediate VIII Via Suzuki-Reaction

Instead of the method of manufacturing Intermediate VIII as described in U.S. Pat. No. 8,609,670 (wherein Intermediate VIII is called Intermediate G):

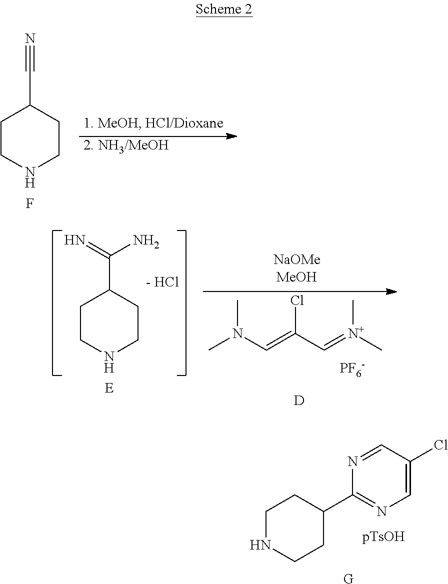

the manufacturing method according to Scheme 3 foresees the manufacture of Intermediate VIII by a Suzuki-reaction:

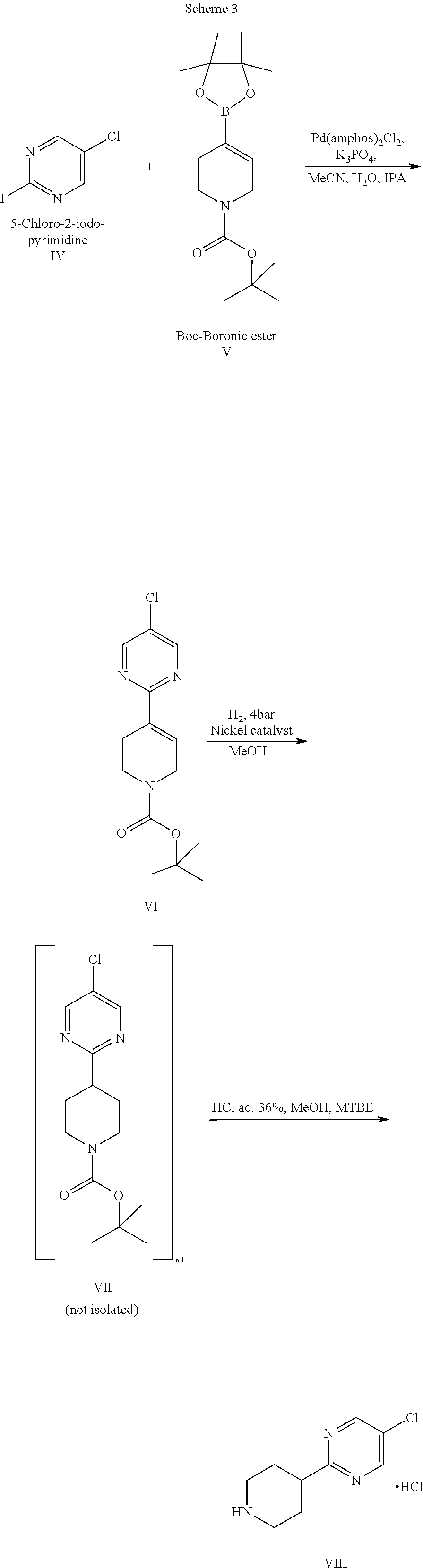

In comparison to U.S. Pat. No. 8,609,670, Intermediate VIII, is manufactured using the starting materials 5-chloro-2-iodo-pyrimidine (IV) and the commodity boc-boronic ester (V). The synthesis includes a palladium-mediated cross-coupling, generating the coupling product VI. The latter compound VI is then hydrogenated to the non-isolated Intermediate VII and treated with hydrochloric acid to form the hydrochloride VIII.

The alternative synthesis method to manufacture Intermediate VIII has several advantages over the method of manufacturing Intermediate VIII (or Intermediate G as it is called in U.S. Pat. No. 8,608,670):
The new Suzuki-reaction process offers much better control of impurities by avoiding the formation of N-methylation at the piperidine ring. The methylation of the piperidine-ring occurs in the original process according to U.S. Pat. No. 8,609,670 during the use of methanol and 4M hydrochloric acid in 1,4-dioxane and takes place via the in situ formed cancerogenic compound methyl chloride. As a result, the Intermediate VIII produced by the original process according to U.S. Pat. No. 8,609,670 leads to a high amount of unwanted N-piperidine methylated side products resulting in a comparably low assay/content and a comparably lower purity of the Intermediate VIII compared to the new Suzuki-reaction.
The cancerogenic solvent 1,4-dioxane can be avoided.
It was surprisingly found that according to the new Suzuki-reaction process only a little amount of the catalyst Pd(amphos)₂Cl₂ can be used. 0.05 m % (0.02 mol %) Pd(amphos)₂Cl₂ is sufficient for the Suzuki cross-coupling reaction to complete the conversion in less than 3 hours. In literature, significant higher amounts of Pd-catalyst have been described so far [a) K.-T.-Wong et al, Org. Lett. 2002, 4 (4), 513; b) P. R. Eastwood, Tetrahedron Lett., 2000, 3705].
The new Suzuki reaction process allows the isolation of Intermediate VIII with a high assay of >98% compared to only 68% as produced according to the original process according to U.S. Pat. No. 8,609,670.
The overall yield to synthesize Intermediate VIII from 5-chloro-2-iodo-pyrimidine (IV) and boc-boronic ester (V) via the new Suzuki reaction process is 68%.
The robustness of the new Suzuki reaction-based process to manufacture Intermediate VIII is beneficial and has been shown on production scale, because first the isolated and non-isolated Intermediates VI and VII are of high stability and stable for storage at room temperature. Second, the reproducibility of yield and quality is very high and consistent as observed on production scale over several batches. Compared to that the original process to manufacture Intermediate VIII according to U.S. Pat. No. 8,609,670 is less controlled, less robust and less reproducible due to the low stability of intermediates E and D
In contrast to the original process as described in U.S. Pat. No. 8,609,670 the new Suzuki reaction based process to manufacture Intermediate VIII involves two catalyst-controlled steps which leads to a complete reaction and hereby to a reduction of the needed amount of educts (leading to lower costs and a lower amount of wastes, an argument that is in particular beneficial for large scale synthesis). Different types of Ni-catalysts with amounts of 6.7-10.0 m % have been used successfully for the hydrogenation step, such as Raney-Nickel and Nickel Sponge. With a lower catalyst load of 6.7 m %, longer hydrogenation times have been observed for full completion on production scale.

### 1.2 Amended Production of the Intermediate XV

Intermediate XV (1-[(2-chloro-6,7-dihydrothieno[3,2-d]pyrimidin-4-yl)amino]-cyclobutanemethanol) has been manufactured according to U.S. Pat. No. 8,609,670 as follows:

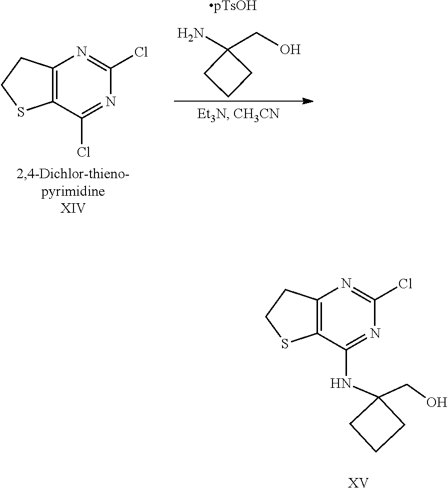

In the amended synthesis according to the present invention Intermediate XV is manufactured as follows (Scheme 4):

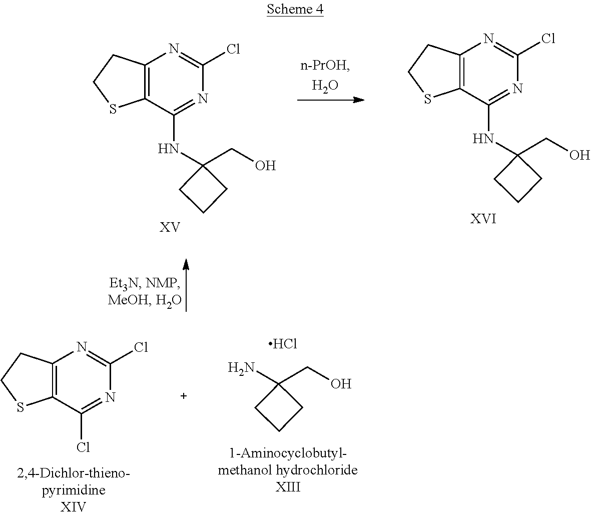

In comparison to the original manufacturing step to yield Intermediate XV according to U.S. Pat. No. 8,609,670 the following aspects have been changed/amended in the corresponding amended manufacturing step to yield Intermediate XV according to the present invention:
The use of 1-aminocyclobutyl-methanol as a hydrochloride salt (Intermediate XIII) instead using the para-toluenesulfonic acid leads to a higher process economy, i.e., less material waste and less salt load in waste streams.
The use of 2.0 volume parts (VP)N-methyl-2-pyrrolidone (NMP) instead of 3.9 VP acetonitrile nearly halves the amount of solvent for the reaction and therefore produces less waste. Volume parts are liter-equivalents of liquids in the ratio of kilogram starting material.
The change of the solvent from acetonitrile to NMP improves the reaction rate substantially from 12 hours down to 6 hours, also by allowing higher reaction temperatures (80° C. instead of 75-77° C.). It was observed that longer reaction times led to increased side reactions (e.g., dimerization) and thereby lowered the possible yield.
The amount of base triethylamine could be reduced to 3.0 equivalents compared to 5.0 equivalents, ensuring full conversion of Intermediate XIV.
Improvement of the reaction conditions (solvent, base, crystallization conditions) allowed isolation of Intermediate XV in higher yield of 73.9% compared to 57%.

### 1.3 Additional Crystallizing Step for Intermediate XV to Yield Intermediate XVI which is Substantially Free of Triethylammonium-Salt (Important to Yield an Enantiomerically Pure Intermediate XVII in the Downstream Step)

This additional crystallizing step of Intermediate XV to yield Intermediate XVI ensures the control of triethylammonium salt of the previous process step by a full depletion. As kilo-lab experiments show, high levels of 10.6% triethylammonium hydrochloride (TEA*HCl) are fully depleted by the crystallization procedure (see example 1 in the below table). The yield loss of the process is nearly fully explained by the amount of triethylammonium hydrochloride (see example 2 in the below table).

These aspects are shown in lab scale experiments (see also chapter synthesis of Intermediate XVI):

| | Process | TEA*HCl content in [%] | | Scale | Yield |
|---|---|---|---|---|---|
| Example | variant | Intermediate XV | Intermediate XVI | [kg] | [%] |
| 1 | A | 10.6 | <0.5 | 2.0 | 83.6 |
| 2 | A | 0.5 | <0.5 | 2.0 | 96.8 |
| 3 | B | 7.8 | <0.5 | 0.05 | 85.4 |
| 4 | B | 0.4 | <0.5 | 0.05 | 96.8 |

The triethylammonium salt-free quality of Intermediate XVI is mandatory to obtain in high robustness enantiomerically pure Intermediate XVII in the downstream stereoselective oxidation step:

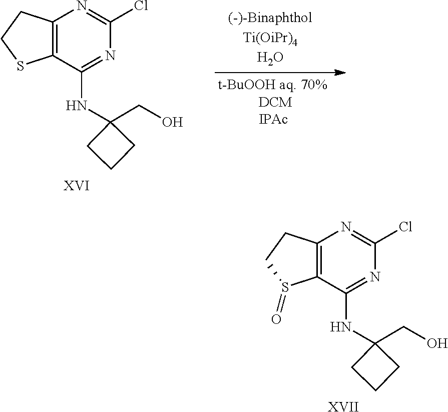

The catalytic cycle of the asymmetric titanium-mediated oxidation is disturbed by the presence of triethylammonium salts.

Additionally, the triethylammonium salt-free quality of Intermediate XVI also allows the isolation of Intermediate XVII with less titanium-byproducts, because the remaining titanium-catalyst is nearly fully purged into the mother- and washing liquor as can be seen from the following experiments (see also chapter synthesis of Intermediate XVII).

| | Compound XVI | Compound XVII | | | |
|---|---|---|---|---|---|
| | TEA*HCl | ent-XVII | Ti content | Scale | Yield |
| Example | [%] | [%] | [mg/kg] | [g] | [%] |
| 1 | <0.5 | <0.06 | 27 | 30.0 | 88.9 |
| 2 | 0.8 | <0.06 | 1080 | 30.0 | 90.0 |
| 3 | 3.4 | 0.39 | 611 | 30.0 | 92.3 |
| 4 | 5.7 | 1.5 | 1293 | 30.0 | 89.7 |
| 5 | 10.6 | 1.7 | 1128 | 30.0 | 86.9 |

The experiments show the correlation between the triethylammonium salt content of Intermediate XVI and the unwanted enantiomeric impurity ent-XVII

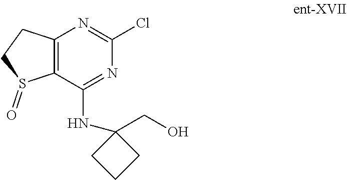 [SMILES: O=[S@]1CCc2nc(Cl)nc(NC3(CO)CCC3)c21]

and the titanium-content of Intermediate XVII. At levels below 0.8% TEA*HCl in Intermediate XVI, the enantiomeric impurity ent-XVII is below detection limit in Intermediate XVII (<0.06) as well as low levels of titanium have been observed at least in example 1 (27 mg/kg). The higher the triethylammonium salt content in Intermediate XVI, the higher the level of the enantiomeric impurity ent-XVII is obtained (see examples 3-5). The described triethylammonium salt free conditions allow the isolation of Intermediate XVII in very high enantiomeric excess of ee>99.88%. In comparison to examples in the literature [a) S. Uemura et al., Tetrahedron Lett. 1992, 33, 5391, b) J. Gao et al., Tetrahedron Lett. 2007, 48, 8453; c) N. Komatsu et al., J. Org. Chem 1993, 58, 4529], which describes enantioselective oxidations of thioethers to sulfoxides using Uemura catalyst [S. Uemura et al., Tetrahedron Lett. 1992, 33, 5391], the enantiomeric purity is higher for the process of the invention.

### More Cost-Efficient Stereoselective Oxidization Step to Yield Intermediate XVII by a New Preincubation Step for S-(−)-Binaphthol in Dichloromethane and the Catalyst Ti(OiPr)₄

In contrast to the description in U.S. Pat. No. 8,609,670 of the stereoselective oxidation step

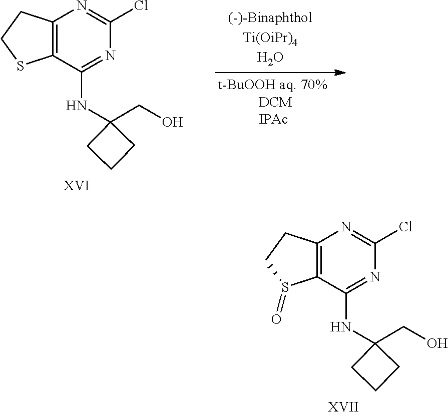

the stereoselective oxidization according to this invention contains an additional preincubation step wherein the S-(−)-binaphthol in dichloromethane and the catalyst Ti(OiPr)₄ are preincubated for about one hour (prior to the addition of Intermediate XVI). Only after that preincubation step the Intermediate XVI is added in an amount that is at least 90-fold (preferably 95-fold, in particular 97-fold) in excess compared to the expensive catalyst Ti(OiPr)₄. According to the description of the corresponding stereoselective oxidization step in U.S. Pat. No. 8,609,670 Intermediate XVI (in U.S. Pat. No. 8,609,670 called compound VIII) was added in the amount of 429 mMol, whereas 21.4 mMol of the expensive catalyst Ti(OiPr)₄ had been used, which means that in U.S. Pat. No. 8,609,670 Intermediate XVI (in U.S. Pat. No. 8,609,670 called compound VIII) was only added in an amount that is 20-fold in excess to the used amount of the catalyst Ti(OiPr)₄. Consequently, the new preincubation step of S-(−)-binaphthol in dichloromethane and the catalyst Ti(OiPr)₄ (prior to the addition of Intermediate XVI) in the stereoselective oxidization step yielding in Intermediate XVII according to the instant application now made it possible to use a clearly reduced amount of the expensive catalyst Ti(OiPr)₄ in comparison to Intermediate XVI. This new preincubation step therefore makes the stereoselective oxidization step yielding in Intermediate XVII more cost-efficient which is particularly important in a large-scale process.

### 1.5 Additional Recrystallization Step of Intermediate XVII after the Stereoselective Oxidation Step to Yield an Intermediate XVIII

In comparison to the manufacturing processes as described in U.S. Pat. No. 8,609,670, the manufacturing process of the invention foresees an additional recrystallization step of Intermediate XVII yielding Intermediate XVIII (as described in detail in Chapter “Synthesis of Intermediate XVIII”) in three variants (variant A: recrystallization from 1,4-dioxane, variant B: recrystallization from a mixture of acetonitrile and water, variant C: recrystallization from tetrahydrofuran):

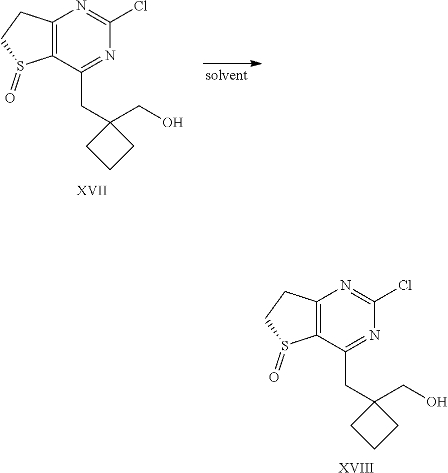

This additional recrystallization step improves the quality of the sulfoxide Intermediate XVII critically.

For example with the additional recrystallization step according to variant A (recrystallization from 1,4-dioxane) Intermediate XVIII is obtained as a water-free form (examples 1 and 2). The water-free material is long-term stable (>12 months) regarding hydrolysis side-reactions, which can occur at two positions of Intermediate XVII by forming IMP-A and IMP-B (see Scheme 5).

In addition, the recrystallization procedure of Intermediate XVII controls the titanium content in the isolated material. In 1,4-dioxane, the titanium-impurities are insoluble and are separated by filtration. The recrystallization ensures titanium levels below 30 ppm.

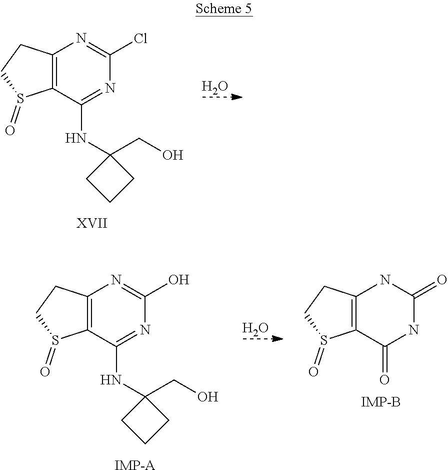

Degradation of Intermediate XVII by hydrolysis.

The table below shows stress test-experiments of the hydrolysis reactions of Intermediate XVII as prepared by the manufacturing process according to the invention. It is shown, that hydrolysis toward IMP-A occurs at 60° C. and 90° C. (examples 1 and 2). The hydrolysis reaction itself is promoted by titanium residues, as example 2 with 671 mg/kg titanium leads to higher impurity levels of IMP-A compared to example 1, which has a lower titanium content (<251 mg/kg).

| | IMP-A [%] | | | IMP-B [%] | | | Ti |
|---|---|---|---|---|---|---|---|
| | XVII at | 1 week at | 1 week at | XVII at | 1 week | 1 week at | content |
| Example | t = 0 | 60° C. | 90° C. | t = 0 | at 60° C. | 90° C. | [mg/kg] |
| 1 | 0.10 | 0.13 | 0.16 | <0.05 | 0.05 | 0.34 | <251 |
| 2 | <0.05 | 0.19 | 0.32 | <0.05 | <0.05 | 0.54 | 671 |

### 1.6 a More Cost-Efficient and More Environment-Friendly Synthesis of Intermediate XIX (in Comparison to the Process as Described in U.S. Pat. No. 8,609,670B)

In comparison to the manufacturing process of Example 2 as described in U.S. Pat. No. 8,609,670B, the corresponding manufacturing step to yield Intermediate XIX

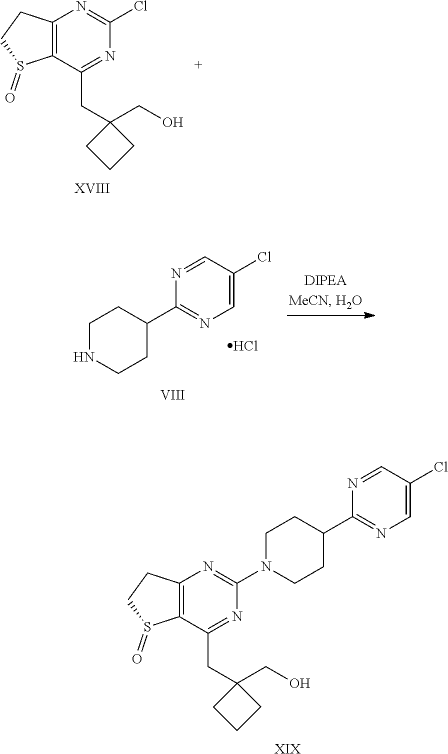

according to the instant invention has been amended as follows:
The amount of N,N-diisopropylethylamine (DIPEA) could be reduced from 2.5 equivalents (eq.) to 2.1 eq., allowing less waste of the ecotoxic base DIPEA
The use of acetonitrile and water as solvents allows surprisingly a fast conversion time of less than 60 min instead of 180 min (as described in U.S. Pat. No. 8,609,670B) which is extraordinarily beneficial in particular in view of large-scale synthesis and process economy. The use of the potential cancerogenic solvent tetrahydrofuran (THF) and acetone as washing medium (as described in U.S. Pat. No. 8,609,670B) could be additionally avoided. It was observed that acetone can be substituted by the green solvent water.

### 1.7 an Improved Final Recrystallization Process with an Improved Conversion Rate from Anhydrous Crystalline Form A to Anhydrous Crystalline Form B (in Comparison to the Process as Described in U.S. Pat. No. 8,609,670B)

In U.S. Pat. No. 8,609,670B also a crystallization process from a mixture of n-propanol and water yielding the PDE4B-inhibitor of formula XX (named Example 2 in U.S. Pat. No. 8,609,670B) in its crystalline form B has been described. However, the crystallization process as described in U.S. Pat. No. 8,609,670B to yield Example 2 in crystalline form B (which is the thermodynamically most stable crystalline form of the PDE4B-inhibitor of formula XX) used a mixture of 9.0 VP n-propanol and 1.0 VP of water as recrystallization solvent. In contrast to that the corresponding recrystallization process of the instant invention makes use of a mixture of 5.0 VP of n-propanol and of 1.25 VP of water. Later, after the formation of the compound of formula XX in its crystalline form B at 40° C., then additional n-propanol (10.0 VP) is added in order to prevent the conversion of crystalline Form B into the dihydrate crystalline Form C.

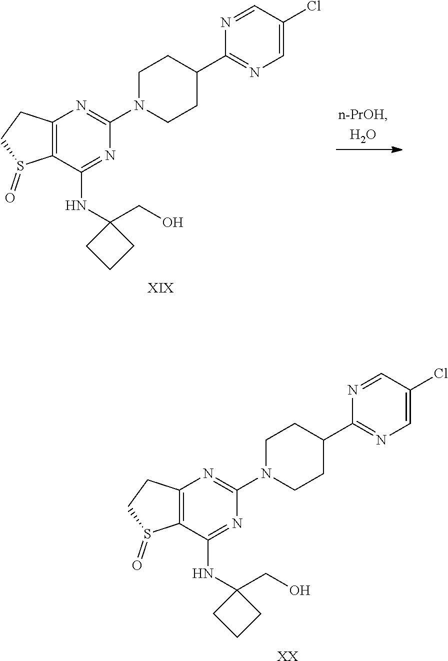

In addition, it was surprisingly observed, that after seeding with seeding crystals of crystalline form B of the compound of formula XX at a temperature of 40° C., the conversion rate of crystalline form A to crystalline form B is significantly enhanced. The temperature of 40° C. allows a full conversion into crystalline form B of the PDE4B-inhibitor of formula XX in at least 4 hours. Therefore, the final crystallization process to yield the PDE4B-inhibitor of formula XX according to this invention is significantly faster from the timepoint of seeding then the corresponding crystallization process known from U.S. Pat. No. 8,609,670B. The crystallization procedure to yield the thermodynamically most stable crystalline form B as described in U.S. Pat. No. 8,609,670B needs at least 13.5 hours (lab scale) from the timepoint of seeding to the timepoint of filtration. However, the crystallization procedure to yield the compound of formula XX as crystalline form B (on production scale) as described in the instant invention (in Chapter 3.10) reduces the crystallization time down to not more than 9 hours, which means a significant improvement in the cycling time and in process economy.

### BRIEF DESCRIPTION OF THE DRAWINGS

FIG. 1a shows the XRPD-analyses of crystalline Form A of the Compound of formula XX (at the top) and of crystalline Form B of the Compound of formula XX and in between the XRPD-analyses of process samples 1a, 2a, 2b and 3a (see Chapter 4.2).

FIG. 1b shows an enlargement of the XRPD-analyses of FIG. 1a in the 2Theta-region of 7 to 12 degrees (see Chapter 4.2). It can be derived from FIG. 1b that the peaks at 2Theta=8.8 degrees and at 2Theta=9.6 degrees which are characteristic for crystalline Form A have completely disappeared in Sample No. 2b (which was isolated after stirring for 2 hours at 40° C. internal temperature) and in Sample No. 3a (which was isolated after stirring for 4 hours at 40° C. internal temperature). Consequently, Sample numbers 2b and 3a consist only of the desired crystalline Form B of the Compound of formula XX, whereas Sample numbers 1a and 2a consist of a mixture of crystalline Forms A and B of the Compound of formula XX.

FIG. 2 shows the conversion rate from crystalline Form A to crystalline Form B of the compound of formula XIX at the different temperatures 20° C., 40° C. and 70° C. In this experiment the Compound of formula XIX has been crystallized from a solution of n-propanol/water after a seeding with 10% of crystalline Form B at 20° C., at 40° C. and at 70° C. FIG. 2 shows the percentage of Form B-formation in dependency of the time and hereby envisions the conversion rates from crystalline Form A to crystalline Form B of the compound of formula XIX at the different temperatures as tested. It can be derived from FIG. 2 that the crystalline Form B-formation is fastest at 40° C., whereas crystalline Form B-formation is comparable at the temperatures of 20° C. and of 70° C., but significantly slower than at 40° C. Consequently 40° C. seems to be the ideal temperature for the formation of the desired crystalline Form B of the Compound of formula XX.

FIG. 3 shows the solubility curves of crystalline Form B and of crystalline Form A of the Compound of formula XX (solvent=13 vol % H₂O in n-propanol).

FIG. 4A describes an analysis of the influence of the water activity of the n-propanol/water mixture on the one hand and of the process temperature on the other hand on crystalline form conversions of the Compound of formula XX, whereas the black line (the “process line”) represents the optimized crystallization process to yield the Compound of formula XX in its crystalline Form B only. The additional late dilution step d8) with 10.0 VP of n-propanol at 20° C./room temperature (which decreases water activity of the solvent from about 0.85 to about 0.65 prior to cooling to 0-5° C. in step d9)) of the optimized crystallization process of the invention leads to the fact that the “process line” in FIG. 4A ends in an area where predominantly Form B of the Compound of formula XX is formed (see black squares around 5° C. and at a water activity of around 0.65), whereas without that late dilution step d8) with 10.0 VP of n-propanol at 20° C./room temperature the “process line” would end in an area where predominantly the unwanted dihydrate Form C of the Compound of formula XX is formed (see grey triangles around water activities around 0.85 and ≤20° C.).

FIG. 4B shows an analysis of the influence of the volume % of water content in n-propanol on the one hand and of the process temperature on the other hand on crystalline form conversions of the Compound of formula XX, whereas the black line (“current process”) represents the optimized crystallization process of the invention to yield the Compound of formula XX in its crystalline Form B only. The additional late dilution step d8) with 10.0 VP of n-propanol at 20° C./room temperature (which decreases the volume % of water in n-propanol from 15 Vol % water to about 7 Vol % water prior to cooling to 0-5° C. in step d9)) of the optimized crystallization process of the invention leads to the fact that the “current process” line in FIG. 4B ends in an area where predominantly Form B of the Compound of formula XX is formed (see black squares around 5° C. and around 7 Vol % of water in nPrOH), whereas without that late dilution step d8) with 10.0 VP of n-propanol at 20° C./room temperature the “current process” line would end in an area where predominantly the unwanted dihydrate Form C of the Compound of formula XX is formed (see grey triangles at ≤20° C. and around 15 Vol % of water in nPrOH).

FIG. 5 shows the powder X-ray diffraction pattern measured by using CuKα radiation of the crystalline anhydrous Form A of the compound of formula

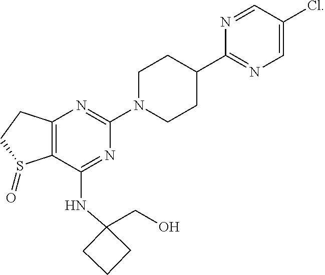 [SMILES: O=[S@@]1CCc2nc(N3CCC(c4ncc(Cl)cn4)CC3)nc(NC3(CO)CCC3)c21]

Form A is basically characterized by XRPD-peaks at the 2Theta values measured by using CuKα radiation: 19.18±0.2; 12.98±0.2; 22.62±0.2; 17.94±0.2 and in particular 8.76±0.2.

FIG. 6 shows the powder X-ray diffraction pattern measured by using CuKα radiation of the crystalline anhydrous Form B of the compound of formula

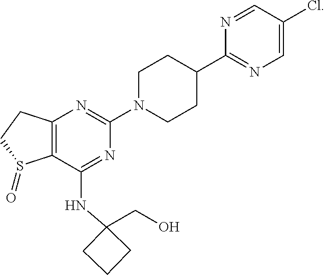 [SMILES: O=[S@@]1CCc2nc(N3CCC(c4ncc(Cl)cn4)CC3)nc(NC3(CO)CCC3)c21]

Form B is basically characterized by XRPD-peaks at the 2Theta values measured by using CuKα radiation: 19.18=0.2; 21.30±0.2; 24.28±0.2; 23.82±0.2 and 4.78±0.2. In particular crystalline Form B does not comprise a 2Theta values measured by using CuKα radiation at 8.76±0.2 (as shown by the experiments shown in Chapter 4.2).

FIG. 7 shows the powder X-ray diffraction pattern measured by using CuKα radiation of the crystalline anhydrous Form B of the compound of formula

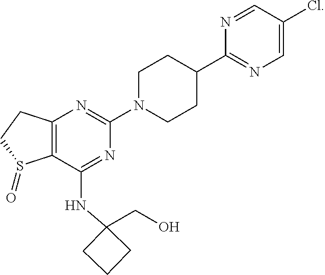 [SMILES: O=[S@@]1CCc2nc(N3CCC(c4ncc(Cl)cn4)CC3)nc(NC3(CO)CCC3)c21]

Form C is basically characterized by XRPD-peaks at the 2Theta values measured by using CuKα radiation: 21.54±0.2; 20.70±0.2; 17.20±0.2; 22.48±0.2 and 26.50=0.2.

FIG. 8 shows the results of a differential scanning calorimetry (DSC) experiment of the Compound of formula XX as manufactured according to the processes as described in the instant invention studied. The Compound of formula XX as manufactured according to the invention showed an endotherm onset at 220.43±2° C. with an enthalpy of 10.5±5 J/g and a peak at 221.29±2° C. The thermal event showed melting and a solid-state phase transformation to the “high temperature Form V” of the Compound of formula XX. The endotherm onset at 237.38±2° C. indicates the melting of the “high temperature Form V” of the Compound of formula XX.

FIG. 9 shows the results of a differential scanning calorimetry (DSC) experiment of a sample of the compound of formula

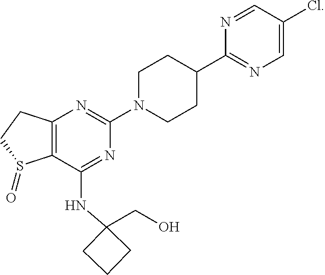 [SMILES: O=[S@@]1CCc2nc(N3CCC(c4ncc(Cl)cn4)CC3)nc(NC3(CO)CCC3)c21]

in its crystalline Form A (as manufactured as described in U.S. Pat. No. 8,609,670B). Crystalline Form A shows a small endotherm at 210.58±2° C. with an enthalpy of 2.08±1 J/g and peak at 212.33±2° C. Thermal events showed solid-state phase transformation to the “high temperature Form V” and the melting of Form V.

### 2 DESCRIPTION OF THE INVENTION

In a first aspect the invention relates to a method of manufacturing the Intermediate VIII

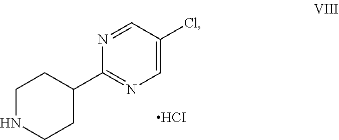

wherein in step a) 5-Chloro-2-iodo-pyrimidine and boc-boronic ester V

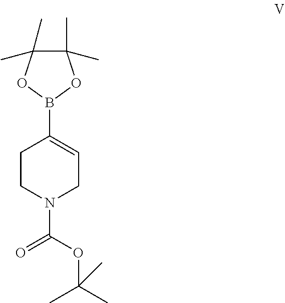 [SMILES: CC(C)(C)OC(=O)N1CC=C(B2OC(C)(C)C(C)(C)O2)CC1]

are reacted in the presence of the catalyst bis(amphos)palladium-II-chloride resulting in Intermediate VI

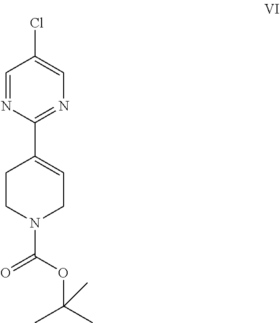 [SMILES: CC(C)(C)OC(=O)N1CC=C(c2ncc(Cl)cn2)CC1]

and wherein in step b) Intermediate VI is hydrogenated to yield the non-isolated Intermediate VII

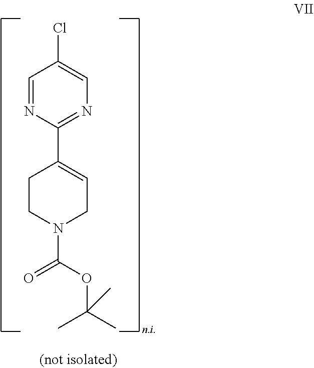

and wherein in step c) the non-isolated Intermediate VII is reacted with hydrochloric acid to yield Intermediate VIII.

In a more preferred embodiment, the above-mentioned reaction step a) is performed in the presence of tri-potassium-phosphate.

In a more preferred embodiment, the above-mentioned reaction step a) is performed in the presence of tri-potassium-phosphate, acetonitrile, water and isopropanol.

In a further preferred embodiment, the above-mentioned reaction step b) is performed in the presence of Raney-Nickel as catalyst.

In a further preferred embodiment, the above-mentioned reaction step b) is performed in the presence of Raney-Nickel as catalyst and in the presence of methanol as solvent.

In a further preferred embodiment, the above-mentioned reaction step c) is performed in the presence of methanol and methyl-tert-butylether.

In a second aspect, the invention relates to a method to manufacture Intermediate XV

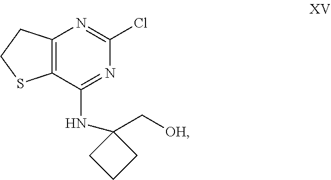 [SMILES: OCC1(Nc2nc(Cl)nc3c2SCC3)CCC1]

wherein 2,4-Dichloro-thienopyrimidine and 1-aminocyclobutyl-methanol hydrochloride are reacted with triethylamine in N-methyl-pyrrolidone at 80° C.

In a preferred embodiment, the invention relates to the above-mentioned method to manufacture Intermediate XV, wherein 2,4-Dichloro-thienopyrimidine and 1-aminocyclobutyl-methanol hydrochloride are reacted with triethylamine in N-methyl-pyrrolidone at 80° C. for 6 hours.

In a particularly preferred embodiment of the above-mentioned method to manufacture Intermediate XV 2,4-Dichloro-thienopyrimidine and 1-aminocyclobutyl-methanol hydrochloride are reacted with triethylamine in only 1.0 to 2.0 VP of N-methyl-pyrrolidone at 80° C.

In a further particularly preferred embodiment of the above-mentioned method to manufacture Intermediate XV 2,4-Dichloro-thienopyrimidine and 1-aminocyclobutyl-methanol hydrochloride are reacted with 3.0 equivalents of triethylamine in 2.0 VP of N-methyl-pyrrolidone at 80° C.

In a third aspect, the invention relates to a method to manufacture an Intermediate XVI

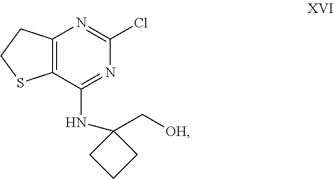 [SMILES: OCC1(Nc2nc(Cl)nc3c2SCC3)CCC1]

which contains ≤0.5% triethylammonium hydrochloride
and which is manufactured
in a first step by the reaction of 2,4-Dichloro-thienopyrimidine and 1-aminocyclobutyl-methanol hydrochloride with triethylamine in N-methyl-pyrrolidone at 80° C. to yield Intermediate XV,
whereby this Intermediate XV yielded from the first step is then in a second step subjected to a crystallization from a solvent mixture to yield Intermediate XVI containing ≤0.5% triethylammonium hydrochloride.

In a preferred embodiment of the above-mentioned method to manufacture Intermediate XVI which contains ≤0.5% triethylammonium hydrochloride

Intermediate XV yielded from the first step is then in a second step subjected to the above-mentioned crystallization step from the solvent mixture of n-propanol and water and involves the following steps:
Intermediate XV yielded from the first step is suspended in a mixture of n-propanol and water at room temperature (preferably under inert atmosphere)
the reaction mixture is then heated to reflux with an internal temperature of 85-95° C. (preferably about 89° C.) and kept at this temperature for at least 30 minutes, then cooled to 60-70° C. internal temperature within at least 30 minutes
after the addition of seeding crystals of Intermediate XVI the reaction mixture is continued stirring at 60-70° C. for at least further 30 minutes,
then water is added to the reaction mixture at an internal temperature of 60-70° C. within at least 60 minutes, stirring is continued at 60-70° C. for at least 30 minutes
then the mixture is cooled to an internal temperature of 15-25° C. (preferably to 20° C.)
the reaction mixture is filtered, washed with water and dried at a temperature≤75° C.

In a particularly preferred embodiment of the above-mentioned method to manufacture Intermediate XVI
which contains ≤0.5% triethylammonium hydrochloride
the above-mentioned crystallization step from a mixture of n-propanol and water involves the following steps:
   Intermediate XV yielded from the first step is suspended in a mixture of 4.0 VP of n-propanol and 7.0 VP of water at room temperature (preferably under inert atmosphere)
   the reaction mixture is then heated to reflux with an internal temperature of about 89° C. and kept at this temperature for at least 30 minutes, then cooled to 60-70° C. internal temperature within at least 30 minutes
   after the addition of seeding crystals of Intermediate XVI the reaction mixture is continued stirring at 60-70° C. for at least further 30 minutes,
   then water is added to the reaction mixture at an internal temperature of 60-70° C. within at least 60 minutes, stirring is continued at 60-70° C. for at least 30 minutes
   then the mixture is cooled to an internal temperature of 20° C. . . . the reaction mixture is filtered, washed with water and dried at a temperature≤75° C.

In another preferred embodiment of the above-mentioned method to manufacture Intermediate XVI
which contains ≤0.5% triethylammonium hydrochloride,
whereby Intermediate XV yielded from the first step is then in a second step subjected to a crystallization from a solvent mixture of 2.5 VP of 1,4-dioxane and of 0.5 VP of water to yield Intermediate XVI containing ≤0.5% triethylammonium hydrochloride.

In a particularly preferred embodiment of the above-mentioned method to manufacture Intermediate XVI
which contains ≤0.5% triethylammonium hydrochloride,
whereby Intermediate XV yielded from the first step is then in a second step subjected to a crystallization from a solvent mixture of 2.5 VP of 1,4-dioxane and of 0.5 VP of water to yield Intermediate XVI containing ≤0.5% triethylammonium hydrochloride, this crystallization step involves the following steps:
   Intermediate XV is suspended in a mixture of 2.5 VP of 1,4-dioxane and of 0.5 VP of water
   the mixture is then heated to 60° C. and kept at this temperature for about 30 minutes.
   2.0 VP of water is added, seeding crystals of Intermediate XVI are added and the suspension is continued stirring at 60° C. for about 30 minutes
   then water is added into the reaction mixture at an internal temperature of 60-70° C. within at least 60 minutes, stirring is continued at 60-70° C. for at least 30 minutes
   then the mixture is cooled to an internal temperature of 15-25° C.
   then the reaction mixture is filtered, washed with water and dried at a temperature about 80° C.

In a fourth aspect, the invention relates to a method to manufacture an Intermediate XVII

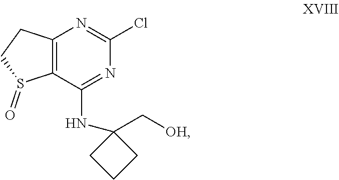 [SMILES: O=[S@@]1CCc2nc(Cl)nc(NC3(CO)CCC3)c21]

which is substantially enantiomerically pure
whereby this substantially enantiomerically pure Intermediate XVII was manufactured by steps a), b) and c),
whereby
step a) involves the reaction of 2,4-Dichloro-thienopyrimidine and 1-aminocyclobutyl-methanol hydrochloride with triethylamine in N-methyl-pyrrolidone (preferably at a temperature between 70-90° C., in particular at a temperature of 80° C.) to yield Intermediate XV

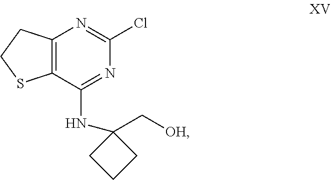 [SMILES: OCC1(Nc2nc(Cl)nc3c2SCC3)CCC1]

step b) involves a crystallization of Intermediate XV yielded in step a) from a mixture of n-propanol and water to yield Intermediate XVI which contains ≤0.5% triethylammonium hydrochloride,
step c) involves the stereoselective oxidation of Intermediate XVI as obtained from step b) with S-(−)-1,1′-Bi-2-naphthol (S-(−)-BINOL), dichloromethane, titanium-(IV)-isopropoxide, water and 70% tert-butylhydroperoxide in water to yield Intermediate XVII.

In a preferred embodiment of the above-mentioned method to manufacture Intermediate XVII this substantially enantiomeric pure intermediate XVII contains ≤0.06% of the unwanted enantiomer ent-XVII

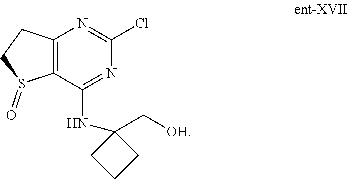 [SMILES: O=[S@]1CCc2nc(Cl)nc(NC3(CO)CCC3)c21]

In a further preferred embodiment of the above-mentioned method to manufacture Intermediate XVII this substantially enantiomerically pure Intermediate XVII has an enantiomeric excess of 99.88% over the unwanted enantiomer ent-XVII.

In a more preferred embodiment of the above-mentioned method to manufacture Intermediate XVII this substantially enantiomeric pure Intermediate XVII contains ≤0.06% of the unwanted enantiomer ent-XVII and contains ≤50 ppm titanium.

In another more preferred embodiment of the above-mentioned method to manufacture Intermediate XVII this substantially enantiomeric pure Intermediate XVII contains ≤0.06% of the unwanted enantiomer ent-XVII and contains ≤30 ppm titanium.

In another more preferred embodiment of the above-mentioned method to manufacture Intermediate XVII this substantially enantiomeric pure Intermediate XVII has an enantiomeric excess of 99.88% and contains ≤50 ppm titanium.

In another more preferred embodiment of the above-mentioned method to manufacture Intermediate XVII this substantially enantiomeric pure Intermediate XVII has an enantiomeric excess of 99.88% and contains ≤30 ppm titanium.

In a fifth aspect, the invention relates to a method to manufacture a substantially enantiomerically pure Intermediate XVIII

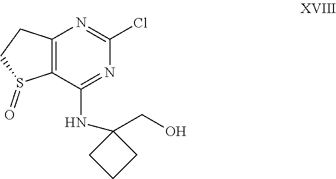 [SMILES: O=[S@@]1CCc2nc(Cl)nc(NC3(CO)CCC3)c21]

which contains <0.06% of the unwanted enantiomer ent-XVII

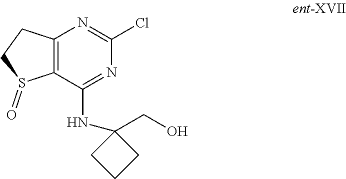 [SMILES: O=[S@]1CCc2nc(Cl)nc(NC3(CO)CCC3)c21]

and which contains ≤30 ppm of titanium,
whereby this substantially enantiomerically pure Intermediate XVII was manufactured by steps a), b), c) and d),
and whereby
step a) involves the reaction of 2,4-Dichloro-thienopyrimidine and 1-aminocyclobutyl-methanol hydrochloride with triethylamine in N-methyl-pyrrolidone (preferably at a temperature between 70-90° C., in particular at a temperature of 80° C.) to yield Intermediate XV

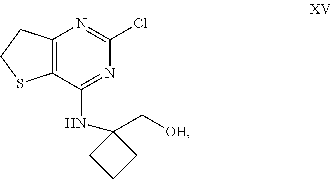 [SMILES: OCC1(Nc2nc(Cl)nc3c2SCC3)CCC1]

step b) involves a crystallization of Intermediate XV yielded in step a) from a mixture of n-propanol and water to yield Intermediate XVI which contains ≤0.5% triethylammonium hydrochloride,
step c) involves the stereoselective oxidation of Intermediate XVI as obtained from step b) with S-(−)-1,1′-Bi-2-naphthol (S-(−)-BINOL), dichloromethane, titanium-(IV)-isopropoxide, water and 70% tert-butylhydroperoxide in water to yield Intermediate XVII, and step d) involves a recrystallization of Intermediate XVII to yield Intermediate XVIII.

In another preferred embodiment of the above-mentioned method to manufacture Intermediate XVIII this substantially enantiomerically pure Intermediate XVIII has an enantiomeric excess over the unwanted enantiomer ent-XVIII of at least 99.88%. In a preferred embodiment of the above-mentioned method to manufacture Intermediate XVIII step d) involves a recrystallization of Intermediate XVII from 1,4-dioxane to yield Intermediate XVIII.

In a more preferred embodiment of the above-mentioned method to manufacture Intermediate XVIII the recrystallization step d) of Intermediate XVII from 1,4-dioxane involves the following steps:
a1) Intermediate XVII is suspended in 8.0 VP 1,4-dioxane
a2) the mixture is heated to 80-90° C. and stirred until complete dissolution
a3) the solution is filtered and concentrated to 3 volume parts
a4) the concentration is stirred for at least 30 minutes
a5) the suspension is slowly cooled to a temperature between 17° C. to 30° C. and stirred
a6) the precipitate is isolated by filtration and the filter cake is washed with acetonitrile or isopropyl acetate
a7) the product is dried to yield Intermediate XVIII.

In a particularly preferred embodiment of the above-mentioned method to manufacture Intermediate XVIII the recrystallization step d) of Intermediate XVII from 1,4-dioxane involves the following steps:
a1′) Intermediate XVII is suspended in 8.0 VP 1,4-dioxane
a2′) the mixture is heated to 80-90° C. and stirred until complete dissolution
a3′) the solution is filtered and concentrated to 3.0 volume parts
a4′) at 70-80° C. seeding crystals of Intermediate XVIII are added to the concentrated solution and the solution is stirred for at least 30 minutes
a5′) the suspension is slowly cooled to a temperature between 22° C. and stirred
a6′) the precipitate is isolated by filtration and the filter cake is washed with acetonitrile
a7′) the product is dried to yield Intermediate XVIII.

In another preferred embodiment of the above-mentioned method to manufacture Intermediate XVIII step d) involve a recrystallization of Intermediate XVII from a mixture of acetonitrile and water to yield Intermediate XVIII.

In a more preferred embodiment of the above-mentioned method to manufacture Intermediate XVIII the recrystallization step d) of Intermediate XVII from a mixture of acetonitrile and water involves the following steps:
b1) Intermediate XVII is suspended in a mixture of 5.0 volume parts (VP) acetonitrile and 2.0 VP water
b2) the mixture is heated to 70-80° C. and stirred until complete dissolution
b3) the solution is filtered using active carbon, the filter is washed with 2.0 VP acetonitrile and concentrated to 3 volume parts
b4) 6.0 VP water are added and the mixture is heated to 70-80° C.
b5) after cooling to 55-65° C. the solution is stirred for at least 1 hour
b6) the precipitate is isolated by filtration and the filter cake is washed with isopropyl acetate
b7) the product is dried to yield Intermediate XVIII.

In a particularly preferred embodiment of the above-mentioned method to manufacture Intermediate XVIII the recrystallization step d) of Intermediate XVII from a mixture of acetonitrile and water involves the following steps:
b1′) Intermediate XVII is suspended in a mixture of 5.0 volume parts (VP) acetonitrile and 2.0 VP water
b2′) the mixture is heated to 70-80° C. and stirred until complete dissolution
b3′) the solution is filtered using active carbon, the filter is washed with 2.0 VP acetonitrile and concentrated to 3 volume parts
b4′) 6.0 VP water are added and the mixture is heated to 70-80° C.
b5′) after cooling to 55-65° C. seeding crystals of Intermediate XVIII are added and the solution is stirred for at least 1 hour
b6′) the precipitate is isolated by filtration and the filter cake is washed with isopropyl acetate
b7′) the product is dried to yield Intermediate XVIII.

It is further preferred that in step b5′) the seeding crystals of Intermediate XVIII are added after cooling to 60° C. and the solution is stirred for at least one hour.

In another preferred embodiment of the above-mentioned method to manufacture Intermediate XVIII step d) involve a recrystallization of Intermediate XVII from tetrahydrofuran to yield Intermediate XVIII.

In a more preferred embodiment of the above-mentioned method to manufacture Intermediate XVIII the recrystallization step d) of Intermediate XVII from tetrahydrofuran involves the following steps:
c1) Intermediate XVII is suspended in 7.0 volume parts (VP) tetrahydrofuran
c2) active carbon is added and the mixture is heated to reflux and stirred for at least 30 minutes
c3) the solution is filtered and concentrated to 2.5 volume parts
c4) the concentrated solution is heated to 50 to 60° C. and the solution is stirred for at least 30 minutes
c5) 2.5 VP isopropyl acetate is added and the solution is stirred for at least 30 minutes
c6) the mixture is cooled to 7 to 13° C.
c7) the precipitate is isolated and then washed with acetonitrile
c8) the product is dried in vacuo to yield Intermediate XVIII.

In a particularly preferred embodiment of the above-mentioned method to manufacture Intermediate XVIII the recrystallization step d) of Intermediate XVII from tetrahydrofuran involves the following steps:
c1′) Intermediate XVII is suspended in 7.0 volume parts (VP) tetrahydrofuran
c2′) active carbon is added and the mixture is heated to reflux and stirred for at least 30 minutes
c3′) the solution is filtered and concentrated to 2.5 volume parts
c4′) at 50-60° C. seeding crystals of Intermediate XVIII are added and the solution is stirred for at least 30 minutes
c5′) 2.5 VP isopropyl acetate is added and the solution is stirred for at least 30 minutes
c6′) the mixture is cooled to 7 to 13° C.
c7′) the precipitate is isolated and then washed with acetonitrile
c8′) the product is dried in vacuo to yield Intermediate XVIII.

It is further preferred that in step c4′) at 55° C. seeding crystals of Intermediate XVIII are added and the solution is stirred for at least 30 minutes.

It is further preferred that in step c6′) the mixture is cooled to 10° C.

It is further preferred that in step c7′) the precipitate is isolated by filtration and the filter cake is washed with acetonitrile.

It is further preferred that in step c2′) 10 m % of active carbon is added.

In a sixth aspect the invention relates to a method of recrystallizing Intermediate XIX to yield the PDE4B-inhibitor of formula XX

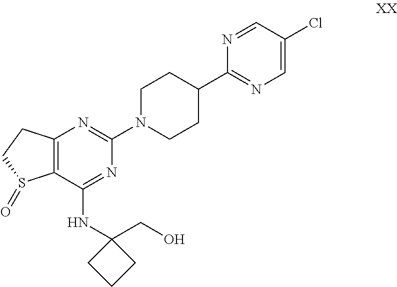 [SMILES: O=[S@@]1CCc2nc(N3CCC(c4ncc(Cl)cn4)CC3)nc(NC3(CO)CCC3)c21]

in the crystalline form B (which is the thermodynamically most stable form and is identical to Form B as described in U.S. Pat. No. 8,609,670B)
   having a powder x-ray diffraction pattern comprising peaks at the following 2 Theta values measured using CuKα radiation: 19.18±0.2; 21.30±0.2; 24.28±0.2; 23.82±0.2 and 4.78±0.2,
   and not comprising peaks at the following 2 Theta values measured using CuKα radiation: 8.76±0.2,
which involves the following steps:
d1) Intermediate XIX is dissolved in a mixture of 5.0 VP of n-propanol and 1.25 VP of water under reflux conditions
d2) the solution is kept at 75-85° C. and filtered
d3) the solution is washed with 2.0 VP of n-propanol
d4) the solution is again heated to reflux and afterwards cooled to 65-75° C.
d5) seeding crystals of Compound XX are added and the mixture is stirred for at least 1 hour (crystalline form A is formed)
d6) the mixture is then cooled to 15-25° C. within at least 90 minutes
d7) after stirring for at least 30 minutes the mixture is heated again to a temperature between 35 to 45° C. (preferably between 37 and 42° C., in particular to 40° C.) within at least 30 minutes to yield crystalline form B of compound XX latest within 4 hours
d8) the mixture is cooled to room temperature and 10.0 VP of n-propanol is added
d9 the mixture is cooled to 0-5° C. and after at least 1 hour compound XX in crystalline form B is isolated
d10) after washing with n-propanol compound XX in crystalline form B is dried.

In a preferred embodiment of the above-mentioned method of recrystallizing Intermediate XIX to yield the PDE4B-inhibitor of formula XX in the crystalline form B in step d2) the solution is kept at 80° C.

In another preferred embodiment of the above-mentioned method of recrystallizing Intermediate XIX to yield the PDE4B-inhibitor of formula XX in the crystalline form B in step d4) the solution is heated again to reflux and afterwards cooled to 70° C.

In another preferred embodiment of the above-mentioned method of recrystallizing Intermediate XIX to yield the PDE4B-inhibitor of formula XX in the crystalline form B in step d6) the mixture is cooled to 20° C. within at least 90 minutes.

In another particularly preferred embodiment of the above-mentioned method of recrystallizing Intermediate XIX to yield the PDE4B-inhibitor of formula XX in the crystalline form B in step d7) after stirring for at least 30 minutes the mixture is heated again to a temperature of 40° C. within at least 30 minutes to yield crystalline form B of compound XX latest within 4 hours.

In a seventh aspect, the invention refers to a method of manufacturing Intermediate XVII

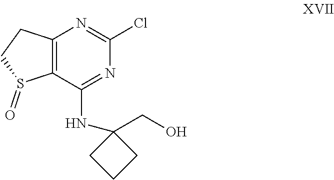 [SMILES: O=[S@@]1CCc2nc(Cl)nc(NC3(CO)CCC3)c21]

by oxidizing the Intermediate XVI

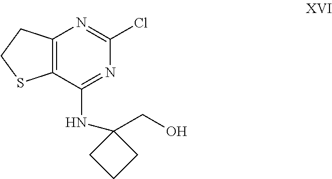 [SMILES: OCC1(Nc2nc(Cl)nc3c2SCC3)CCC1]

in the presence of S-(−)-Binaphthol, Ti(OiPr)₄ and t-BuOOH with a reduced amount of the catalyst Ti(OiPr)₄, wherein this method involves the following steps e1) to e5)
   e1) suspension of S-(−)-binaphthol in dichloromethane under inert atmosphere,
   e2) addition of Ti(OiPr)₄ to the suspension and preincubation of this mixture for at least 1 hour,
   e3) after that preincubation an amount of Intermediate XVI is added to the mixture that is >90-fold in excess to the amount of the catalyst Ti(OiPr)₄ and the suspension is incubated for at least one hour,
   e4) then t-BuOOH is added and the mixture is stirred until reaction completion
   e5) the precipitate containing Intermediate XVII is isolated.

In a particularly preferred embodiment of the above-mentioned method of manufacturing Intermediate XVII the t-BuOOH in step e4) is added portion wise and the mixture is stirred until reaction completion.

### 3 METHOD OF SYNTHESIS

### 3.1 Synthesis of Intermediate IV

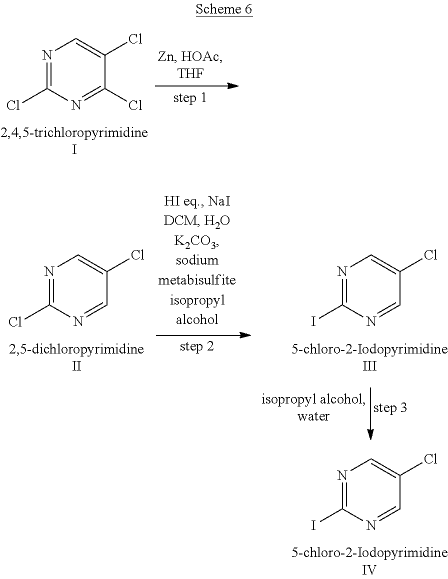 [SMILES: Clc1ncc(Cl)c(Cl)n1 ; Clc1cnc(Cl)nc1 ; Clc1cnc(I)nc1 ; Clc1cnc(I)nc1]

### 3.1.1 Process Description of Step 1 (Intermediate II)

THF (960 mL), 2,4,5-trichloropyrimidine I (1.0 eq., 120.0 g) and acetic acid (58.9 g) were charged to a reactor at room temperature under nitrogen atmosphere. Under stirring, the reaction solution was heated to 40-45° C. Zinc powder (2.0 eq. 85.6 g) was added portion-wise within 2 hrs and the reaction mixture is continued stirring for 16-24 hrs at a temperature of 40-45° C. Upon completion of the reaction, the mixture was cooled to 30-35° C. and Kieselguhr (12 g) and dichloromethane (480 mL) were added. Then the mixture was continued stirring for 30 min, cooled to 15-25° C., filtered and the cake was rinsed with dichloromethane (120 mL). The combined filtrates were distilled at 30-35° C. under vacuum (200-300 mbar) to 2-3 VP. Then dichloromethane (240 mL) was charged, and the organic solution was washed with aqueous citric acid solution (10 wt %, 1200 g). Phases were separated and the aqueous phase was back extracted two times with dichloromethane (480 mL+240 mL). The combined organic layers were washed with aqueous sodium hydrogen carbonate solution (5 wt %, 600 g) and then with water (2×240 mL). Activated charcoal (6.0 g) was charged and the mixture was stirred for 1 h at 20-30° C., filtered and the cake was washed with dichloromethane (120 mL). The filtrate was distilled at 30-35° C. under vacuum (200-500 mbar) to 0.8-1.2 V. Then ethanol (120 mL) was charged, and solvents were distilled at 35-40° C. under vacuum (100-200 mbar) to 0.8-1.2 VP. The resulting solution was cooled to 24-28° C., seeding crystals (0.1 g) were added and the mixture was continued stirring for 20-25° C. for 1 h. The suspension was then cooled down to −5 to ˜10° C. in 2 hrs and continued stirring for at least 3 hrs. The product was filtered, and Intermediate II was obtained in 54% yield (52.6 g). Drying was not carried out due to sublimation of the compound.

### 3.1.2 Process Description of Step 2 (Intermediate III)

Under nitrogen and stirring, hydroiodic acid (55-57 wt %, 2000 g) and then sodium iodide (201 g, 1.0 eq) and Intermediate II (200 g) were charged into a reactor at −2° C. to 2° C. and continued stirring under these conditions for 16 hrs. Upon completion of the reaction, the mixture was filtered at 0-10° C. and then the aqueous filtrate was extracted with dichloromethane (400 mL). The filter cake and the dichloromethane extract were combined, and water (800 mL) was charged. Potassium carbonate solution (50 wt % aqueous solution, 290 g) was charged at 0-10° C. to reach pH value within 6.5-7.5. Then sodium metabisulfite solution (30 wt % aqueous solution, 26 g) was added at 0-10° C. The phases were separated, and the aqueous phases were extracted with dichloromethane (2×400 mL). The combined organic phases were washed with water (400 mL), filtered to remove insoluble material, and then distilled at 30-40° C. under vacuum (300-600 mbar) to 2-3 VP. Then isopropanol (1200 mL) was added and distilled at 50-55° C. under vacuum (150-300 mbar) to 4-5 VP. For crystallization, water (1200 mL) was charged to the solution at 50-55° C. in 2-3 hrs, and the resulting mixture was cooled to 20-25° C. within 2-3 hrs and stirred at this temperature for at least 2 hrs. The product was isolated by filtration, the filter cake washed with a mixture of isopropanol/water (1/2, w/w) (200 g) and drying at 20-25° C. under 50-100 mbar afforded Intermediate III in 74% yield (238 g).

### 3.1.3 Process Description of Step 3 (Intermediate IV)

To a reactor and under nitrogen atmosphere were charged Intermediate III (20 g) and isopropanol (20 mL) and heated to 45-50° C. to form a slurry. Then, water (30 mL) was added within 2-3 hrs. The slurry was cooled to 20-25° C. in 2-3 hrs and stirred at this temperature for another 2 hrs. The suspension was filtered, and the filter cake was washed with a mixture of isopropanol/water (1/2, w/w) (20 g) and drying at 20-25° C. under 50-100 mbar afforded Intermediate IV in 93% yield (18.6 g).

### 3.2 Synthesis of Intermediate VI

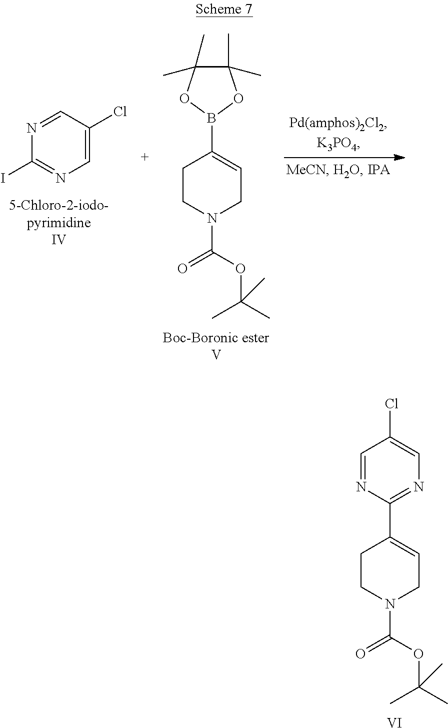

102 kg 5-Chloro-2-iodo-pyrimidine (IV) and 128 kg Boc-boronic ester (V) were charged into a vessel with a stirrer under inert conditions followed by the addition of acetonitrile (202 kg) and isopropanol (199 kg). A solution of tri-potassium phosphate (132 kg) dissolved in water (512 kg) was added. After inertisation the catalyst bis(amphos)palladium-II-chloride (0.05 m %) was added. The mixture was heated under reflux for 3 h. At 70° C. N-acetyl-L-cysteine was charged (2.6 kg, 2.0 m %) and the mixture stirred for 30 minutes at this temperature. Water (576 kg) was added at 70° C. and at ca. 55° C. seeding (0.1 m % seed crystals) was carried out. After cooling to 20° C. the mixture was stirred for 1 h. The resulting solid was collected by centrifugation and washed with water. The product was dried in vacuo in an agitated dryer at 60° C. to afford Intermediate VI as a white solid in 90.9% yield.

¹H NMR (500 MHZ, DMSO-d₆) δ=8.90 (s, 2H), 7.18 (br s, 1H), 4.10 (br s, 2H), 3.57-3.49 (m, 2H), 2.63-2.52 (m, 2H), 1.43 (s, 9H) LCMS (ESI pos) for C₁₄H₁₈ClN₃O₂, (M+H)+ calcd. 296.1, measd. 296.1.

### 3.3 Synthesis of Intermediate VIII

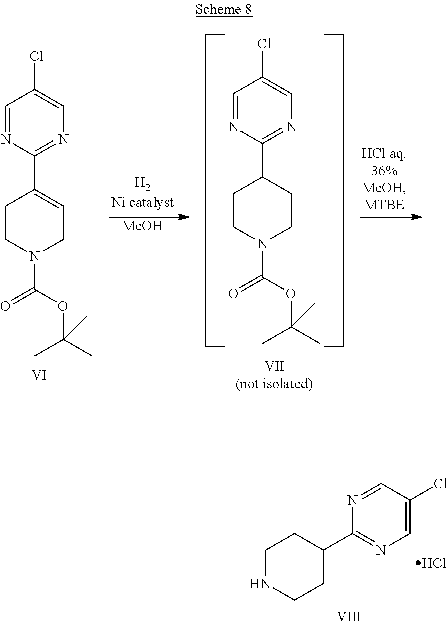

Intermediate VI (111.3 kg) was dissolved in methanol (571 kg) in a hydrogenation vessel. After addition of the Raney-Nickel (10 m %)-catalyst the hydrogenation (4 bar) was carried out at 50° C.

After completion, the catalyst was filtered off and the reaction solution with Intermediate VII was concentrated in vacuo at 70° C. The solution was cooled to 20° C. and added to hydrochloric acid (57 kg) in methanol (160 kg) under reflux conditions. After stirring for 1 h the MeOH/HCl mixture is distilled off under vacuum at 75° C. MtBE (493 kg) was slowly charged to the solution at 60° C. and the mixture was stirred under reflux conditions for 30 min. For crystallization the mixture was cooled to 20° C. and stirred for 1 h. The resulting solid was collected by centrifugation and washed with MtBE to afford Intermediate VIII as a white solid in 74.3% yield after agitated drying at 60° C. in vacuo (HPLC-assay: >98%).

### Compound VII (not Isolated)

¹H NMR (500 MHZ, DMSO-d₆) δ=8.88 (s, 2H), 4.09-3.92 (m, 2H), 3.03 (tt, J=3.7, 11.6 Hz, 1H), 2.88 (br s, 2H), 1.92 (br dd, J=2.0, 12.9 Hz, 2H), 1.65-1.50 (m, 2H), 1.41 (s, 9H). LCMS (ESI pos) for C₁₄H₂₀ClN₃O₂, (M+H)+ calcd. 298.1, measd. 298.1

### Compound VIII

¹H NMR (500 MHZ, DMSO-d₆) δ=8.95 (br s, 1H), 8.92 (s, 2H), 3.34-3.27 (m, 2H), 3.18 (tt, J=3.7, 11.3 Hz, 1H), 3.03 (dt, J=3.0, 12.6 Hz, 2H), 2.12 (br dd, J=2.4, 13.9 Hz, 2H), 2.01-1.89 (m, 2H).

LCMS (ESI pos) for C₉H₁₂ClN₃, (M+H)+ calcd. 198.1, measd. 198.1

In contrast to the manufacturing method as described in U.S. Pat. No. 8,609,670B, Intermediate VIII is manufactured according to the invention using the starting materials 5-chloro-2-iodo-pyrimidine (Intermediate IV) and the commodity Boc-boronic ester (Intermediate V). The synthesis includes a palladium-mediated cross-coupling, generating the coupling product VI.

The latter Intermediate VI is then hydrogenated to the non-isolated Intermediate VII and treated with hydrochloric acid to form the Intermediate VIII as hydrochloride.

This new manufacturing process for the production of the Intermediate VIII as hydrochloride according to the invention (Suzuki reaction) combines several advantages in comparison to the synthesis disclosed in U.S. Pat. No. 8,609,670B:
The new Suzuki type reaction process according to the instant invention offers a much better control of impurities by avoiding the formation of N-methylation at the piperidine ring. The methylation of the piperidine-ring occurs in the process according to U.S. Pat. No. 8,609,670B during the use of methanol and 4M hydrochloric acid in 1,4-dioxane and takes place via the in situ formed cancerogenic compound methyl chloride. Examples 1, 2 and 3 as described in Table A show the analysis of three large scale manufacturing batches of Intermediate VIII which all three have been manufactured according to the new Suzuki reaction type process as described in detail in Chapters 3.2 and 3.3 of the instant application. In all three manufacturing batches Examples 1, 2 and 3 the amount of the N-methylates derivatives of Intermediate VIII (which is an undesired impurity) could not be measured/was below the limit of detection) resulting in a high HPLC purity of usually over 99%:

TABLE A

| | HPLC Purity | HPLC-assay | Scale | Yield |
|---|---|---|---|---|
| Example | [%]* | [%] | [kg] | [%] |
| 1 | 99.45 | 99.8 | 32.7 | 68.8 |
| 2 | 99.71 | 99.7 | 33.2 | 69.9 |
| 3 | 99.85 | 100.2 | 35.7 | 75.2 |

In contrast to that, Examples 1, 2 and 3 as described in Table B show the corresponding analysis of three other large scale manufacturing batches of Intermediate VIII which all three have been manufactured according to the manufacturing process to yield Intermediate C (identical to Intermediate VIII of the instant application) as described in U.S. Pat. No. 8,609,670B:

TABLE B

| | | Impurity N- | | | |
|---|---|---|---|---|---|
| | | methylated | | | |
| | | compound | | | |
| | HPLC Purity | VIII, HPLC | NMR-assay | Scale | Yield |
| Example | [%] | [%] | [%] | [kg] | [%]** |
| 1 | 92.0 | 7.0 | 43.6 | 38.0 | 65.0 |
| 2 | 90.0 | 5.1 | 40.2 | 38.0 | 75.7 |
| 3 | 88.8 | 8.7 | 41.1 | 38.0 | 68.7 |

As can be derived from Table B the manufacturing process to yield Intermediate C (identical to Intermediate VIII of the instant application) as described in U.S. Pat. No. 8,609,670B leads to amounts of approx. 5.1 to 8.7% of N-methylated derivatives of Intermediate VIII as undesired impurities.
The cancerogenic solvent 1,4-dioxane can be avoided by the Suzuki reaction.
It was surprisingly found, that during the Suzuki reaction little amount of the catalyst Pd(amphos)₂Cl₂ can be used. 0.05 mass percent (m %) (=0.02 mol %) Pd(amphos)₂Cl₂ is sufficient for the cross-coupling reaction to complete the conversion in less than 3 hours. In literature, significant higher amounts of Pd-catalyst have been described so far [a) K.-T.-Wong et al, Org. Lett. 2002, 4 (4), 513; b) P. R. Eastwood, Tetrahedron Lett., 2000, 3705].
The new Suzuki type reaction process allows the isolation of Intermediate VIII with a high assay of >98% compared to only an assay of 68% as described in U.S. Pat. No. 8,609,670B.
The all over yield is 68% to synthesize VIII from 5-chloro-2-iodo-pyrimidine (IV) and boc-boronic ester (V). This is the same yield as in the process using 4-cyanopiperidine as starting material in U.S. Pat. No. 8,609,670B.
The robustness of the Suzuki type reaction process is beneficial and has been shown on production scale, because first the isolated and non-isolated Intermediates VI and VII are of high stability and stable for storage at room temperature. Second, the reproducibility of yield and quality is very high and consistent as observed on production scale over several batches.
Different types of Ni-catalysts with amounts of 6.7-10.0 m % have been used successfully for the hydrogenation step, such as Raney-Nickel and Nickel Sponge. With a lower catalyst load of 6.7 m %, longer hydrogenation times have been observed for full completion on production scale.

### 3.4 Synthesis of Intermediate XIII

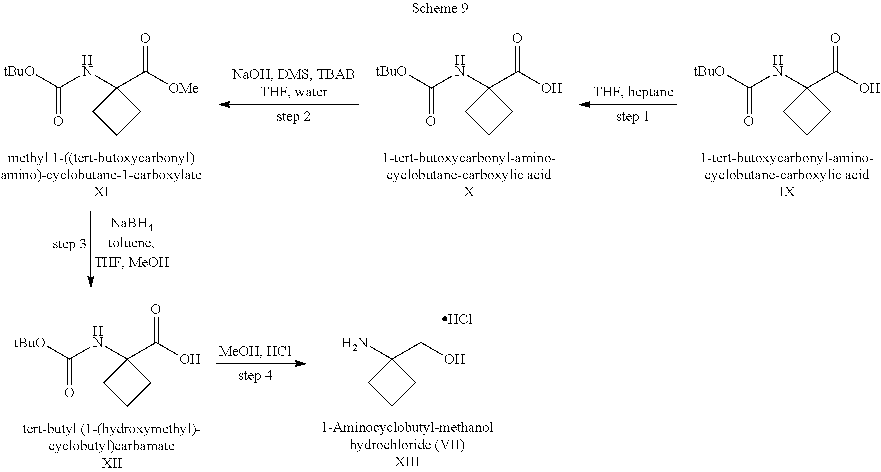 [SMILES: COC(=O)C1(NC(=O)OC(C)(C)C)CCC1 ; CC(C)(C)OC(=O)NC1(C(=O)O)CCC1 ; CC(C)(C)OC(=O)NC1(C(=O)O)CCC1 ; CC(C)(C)OC(=O)NC1(C(=O)O)CCC1 ; NC1(CO)CCC1 ; Cl]

### 3.4.1 Process Description of Step 1 (Intermediate X)

Compound IX (255 kg) and THF (905 kg) were charged into a glass-lined reactor under nitrogen atmosphere at 20° C. The solution was filtered and collected. The cake was washed with THF (112.2 kg) and the filtrates were combined. The filtrates were warmed to 40-50° C. and concentrated under reduced pressure to a residual solvent volume of 1.8-2.0 VP. Heptane (1734 kg) was charged into the reactor at 40-50° C. and the mixture was stirred for 30-60 min at 40-50° C. The mixture was cooled down to 5-15° C. and stirred for another 60-120 min. The product was isolated by centrifugation and the filter cake was washed with heptane (173.4 kg). The solid was dried under vacuum at a temperature of max. 50° C. to afford Intermediate X in 91% yield (233 kg).

### 3.4.2 Process Description of Step 2 (Intermediate XI)

Water (279.6 kg) and NaOH (93.2 kg) were charged into a preparation vessel and stirred until full dissolution. Intermediate X (233 kg) and THF (1239.6 kg) were charged into a suitable inert reactor. Tetrabutylammonium bromide (TBAB) (4.7 kg) was charged into the reactor and the reactor was inertized with nitrogen. The reaction mixture was cooled down to 5-10° C. while stirring. The sodium hydroxide solution was added to the reactor at 5-15° C., then stirred for 1-2 h. Dimethyl sulfate (293.6 kg) was charged into the above reaction mass at 5-15° C. The reaction mixture was stirred for 13-14 h at 20-30° C. After completion of the reaction the mixture was left standing at 20-30° C. for 0.5-1.5 h. The bottom aqueous layer was removed. MgSO₄ was charged into the reactor and the mixture was stirred at 25-30° C. for 1-2 h, then the suspension was filtered, and the filtered cake was washed with toluene (349.5 kg). The filtrate and the toluene wash were combined to give a solution of Intermediate XI and this solution was directly applied for the next synthesis stage.

### 3.4.3 Process Description of Step 3 (Intermediate XII)

Sodium borohydride (107 kg) and toluene (1738 kg) were charged into an inert reactor. The mixture was stirred for 20-30 min at 20-30° C. The THF/toluene solution (1700 kg) of Intermediate XI and methanol (190 kg) were charged into a second reactor and the internal temperature of the mixture was adjusted to 20-30° C. Then, the solution of Intermediate XI in THF/toluene/methanol was added to the solution of sodium borohydride in toluene at 20-30° C. and the reaction mixture was stirred for 12-14 h at 20-30° C. After completion of the reaction, pH of the reaction mixture was adjusted to 7.0-7.5 by adding of hydrochloric acid 36% (168 kg) while maintaining the temperature at 10-20° C. The reaction mixture was filtered, and the cake was washed with ethyl acetate (216 kg). The temperature of the filtrates was adjusted to 50-55° C. and the reaction solution was concentrated under reduced pressure to a residual solvent volume of 0.9-1.1 VP. Then the residual solution was cooled to 20-25° C. and ethyl acetate (1298.4 kg) and water (840 kg) were added. The mixture was stirred at 20-30° C. for 30-40 min and left stand at 20-30° C. for 30-40 min. The layers were separated. The aqueous layer was extracted two times with ethyl acetate (2×432 kg) and the combined organic phases were concentrated at 50-55° C. under reduced pressure to a residual solvent volume of 0.9-1.1 VP. Then heptane (492 kg) was charged at 45-55° C. and the mixture was continued stirring for 0.5 h and then cooled down to 5-10° C. for 0.5-1 h, and the suspension stirred for further 1-2 h at 5-10° C. The suspension was then isolated by filtration, washed with heptane (247.2 kg) and dried under vacuum at 40-45° C. to afford Intermediate XII in 80% yield (174 kg).

### 3.4.4 Process Description of Step 4 (Intermediate XIII)

Intermediate XII (172.8 kg) and methanol (547.8 kg) were charged into a reactor at 25-30° C. At an internal temperature of 20° C., hydrochloric acid 36% (176.3 kg) is slowly added while maintaining the temperature at 20-30° C. Upon completion, the reaction mass temperature was slowly increased to 50-55° C. and stirred for 3-4 hours at 50-55° C. After completion of the reaction, solvents were distilled off under reduced pressure and internal temperature of 45-50° C. to a residual solvent volume of 0.9-1.0 VP. Then isopropanol was added (5×406.1 kg) and solvents were distilled off under reduced pressure to a residual solvent volume of 0.9-1.0 VP. Upon completion of the distillations, the reactor content was cooled down to 20-30° C. for 0.5-1 h. MtBE (767.2 kg) was charged to the reactor and the mixture was cooled to 5-10° C. Intermediate XIII was isolated by filtration, washed with MtBE (127.9 kg), dried under vacuum at a temperature of 40-50° C., and obtained in 94% yield (111 kg).

### 3.5 Synthesis of Intermediate XV

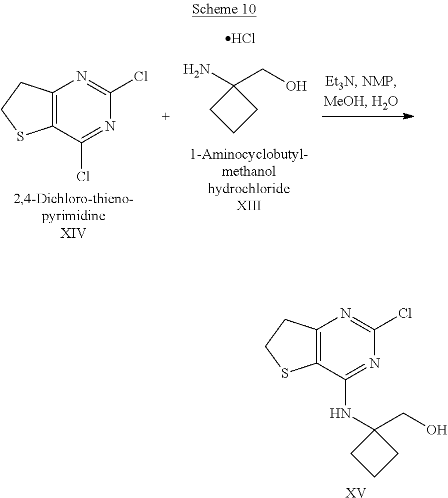

66.3 kg 2,4-Dichloro-thieno-pyrimidine (XIV) and 48.5 kg 1-aminocyclobutyl-methanol hydrochloride (XIII) were charged into a vessel under inert atmosphere and 136 kg N-methyl-pyrrolidone were added. 32.4 kg Triethylamine (1 eq) were added, and the mixture was heated and stirred. The remaining triethylamine amount (64.8 kg) was slowly added in another two portions while the mixture was stirred at 80° C. After completion within 6 hours, the reaction mixture was cooled to 60° C. and methanol (262 kg) were added. The reactor content was charged to a vessel containing 52 kg warm methanol which is then heated to reflux condition. Seeding crystals (1.0 m %) were added after cooling to 55° C. To complete the crystallization the mixture is cooled to 0° C. and stirred for ca. 3 h. The product is isolated by filtration and the filter cake is washed with water and methanol to afford Intermediate XV in 73.9% yield after drying at 60° C. in vacuo in the filter dryer.

¹H NMR (500 MHZ, DMSO-d₆) δ=7.10 (s, 1H), 4.82 (t, J=5.8 Hz, 1H), 3.67 (d, J=5.8 Hz, 2H), 3.36-3.29 (m, 2H), 3.17-3.05 (m, 2H), 2.24-2.10 (m, 4H), 1.82-1.67 (m, 2H). LCMS (ESI pos) for C₁₁H₁₄ClN₃OS, (M+H)+ calcd. 272.1, measd. 272.1

In comparison to U.S. Pat. No. 8,609,670B the following aspects have been amended:
the use of 1-aminocyclobutyl-methanol as a hydrochloride salt (XIII) instead using the corresponding para-toluenesulfonic acid salt leads to a higher process economy, i.e., less material waste and less salt load in waste streams.
the use of 2.0 VP N-methyl-2-pyrrolidone (NMP) instead of 3.9 VP acetonitrile nearly halves the amount of solvent for the reaction and therefore produces less waste. The change of the solvent from acetonitrile to NMP improves the reaction rate substantially from 12 hours down to 6 hours, also by allowing higher reaction temperatures (80° C. instead of 75-77° C.). It was observed that longer reaction times led to increased side reactions (e.g., dimerization) and thereby lowered the possible yield.
the amount of base triethylamine could be reduced to 3.0 equivalents compared to 5.0 equivalents, ensuring full conversion of Intermediate XIV.
the reaction conditions had been improved (solvent, base, crystallization conditions) and therefore allowed the isolation of Intermediate XV in a higher yield of 73.9% compared to 57% (as according to the process as described in U.S. Pat. No. 8,609,670B).

### 3.6 Synthesis of Intermediate XVI

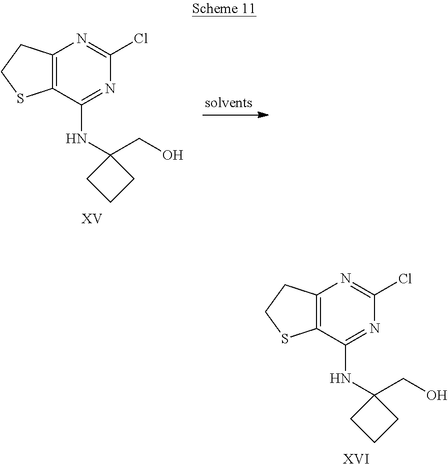

### Variant A:

2.0 kg of Intermediate XV were charged into a vessel under inert atmosphere and room temperature and suspended in n-propanol (4.0 kg) and water (7.0 kg). Then the mixture was heated to reflux (approx. 89° C. internal temperature; jacket temperature: 110° C.) and kept at this temperature for 30 min. Under these conditions the suspension turned into a clear solution. Then the solution was cooled to 60-70° C. internal temperature within 30 min and seeding crystals of Intermediate XVI (2 g) were added. The suspension was continued stirring at 60-70° C. for 30 min. Then, water (8 kg) was dosed into the reaction solution at an internal temperature of 60-70° C. within 60 min. The reactor content was continued stirring at an internal temperature of 60-70° C. for 30 min and then cooled within 60 min to an internal temperature of 20° C. The product was isolated by filtration, washed with water (8 kg) and dried at max. 75° C. to afford Intermediate XVI in a yield of 83.6% (1.67 kg).

¹H NMR (500 MHZ, DMSO-d₆) δ=7.10 (s, 1H), 4.82 (t, J=5.8 Hz, 1H), 3.67 (d, J=5.8 Hz, 2H), 3.36-3.29 (m, 2H), 3.17-3.05 (m, 2H), 2.24-2.10 (m, 4H), 1.82-1.67 (m, 2H).

LCMS (ESI pos) for C₁₁H₁₄ClN₃OS, (M+H)+ calcd. 272.1, measd. 272.1

### Variant B:

50 g of Intermediate XV were charged into a vessel under inert atmosphere and room temperature and suspended in 1,4-dioxane (125 mL, 2.5 VP) and water (25 g, 0.5 VP). Then the mixture was heated to 60° C. and kept at this temperature for 30 min. Under these conditions water (100 g, 2.0 VP) is dosed within 30 min. Then seeding crystals of Intermediate XVI (0.05 g) were added and the suspension was continued stirring at 60° C. for 30 min. Then, water (250 g, 5.0 VP) is dosed into the reaction solution within 1 hour and the crystal suspension is stirred for another 30 min at 60° C. Then, the suspension is cooled within 68 min to 20° C. and stirred for 67 min. The product was isolated by filtration, washed with water (250 g, 5.0 VP) and dried at 80° C. under reduced pressure to afford Intermediate XVI with a yield of 96.8% (48.4 g).

Other solvent systems, which are applicable for the recrystallization of intermediate XV yielding triethylammonium hydrochloride (TEA*HCl) free Intermediate XVI are e.g.: methanol/water, ethanol/water, iso-propanol/water, n-butanol/water, tert-butanol/water, acetonitrile/water, tetrahydrofuran/water, n-methylpyrrolidon/water, dimethylsulfoxide/water and dimethylformamide/water. In terms of processability and yield, the described solvent systems n-propanol/water (variant A) and dioxane/water (variant B) are more preferred.

This additional crystallizing step yielding Intermediate XVI (which was not performed according to the processes as described in U.S. Pat. No. 8,609,670B) ensures the control of triethylammonium salt as an unwanted impurity (that has been produced in the previous manufacturing step according to Scheme 6) by a full depletion. As kilo-lab experiments show, high levels of 10.6% Triethylammonium hydrochloride (TEA*HCl) are fully depleted by the above-described crystallization procedure of variant A from propanol and water (see example 1 in the table). The yield loss of the process is nearly fully explained by the amount of triethylammonium hydrochloride (see example 2 in the table: according to example 2 the yield was 96.8%, according to example 1 the yield was 83.6%, the difference in yield (13.2% seems to consist basically of the impurity TEA*HCl).

As kilo-lab experiments show, high levels of 7.8% Triethylammonium hydrochloride (TEA*HCl) are fully depleted by the above-described crystallization procedure of variant B from 1,4-dioxane (125 mL, 2.5 VP) and water (25 g, 0.5 VP) (see example 3 in the table). The yield loss of the process is partially explained by the amount of triethylammonium hydrochloride (see example 4 in the table: according to example 4 the yield was 96.8%, according to example 3 the yield was 85.4%, the difference in yield (11.4% seems to consist at least partially of the impurity TEA*HCl).

| | Process | TEA*HCl content in [%] | | Scale | Yield |
|---|---|---|---|---|---|
| Example | variant | Intermediate XV | Intermediate XVI | [kg] | [%] |
| 1 | A | 10.6 | <0.5 | 2.0 | 83.6 |
| 2 | A | 0.5 | <0.5 | 2.0 | 96.8 |
| 3 | B | 7.8 | <0.5 | 0.05 | 85.4 |
| 4 | B | 0.4 | <0.5 | 0.05 | 96.8 |

Further, the triethylammonium salt-free quality of Intermediate XVI is mandatory to obtain in high robustness enantiomerically pure Intermediate XVII in the downstream stereoselective oxidation step. The catalytic cycle of the asymmetric titanium-mediated oxidation is disturbed by the presence of triethylammonium salts. Additionally, the triethylammonium salt free quality of Intermediate XVI also allows the isolation of Intermediate XVII with less titanium-byproducts, because the remaining titanium-catalyst is nearly fully purged into the mother—and washing liquor.

These aspects are shown in lab scale experiments (see chapter “3.7 Synthesis of Intermediate XVII”).

### 3.7 Synthesis of Intermediate XVII

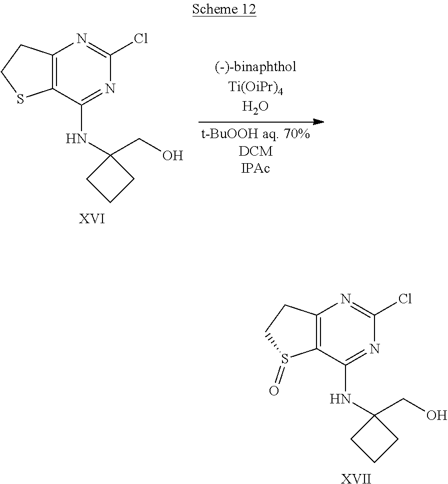

2.03 kg S-(−)-binaphthol were charged into a vessel under inert atmosphere and suspended in 192 kg dichloromethane. After addition of 1,038 kg titanium-(IV)-isopropoxide (3.6522 mol) and 1.274 kg water the mixture was stirred for ca. 1 h (preincubation step). After that preincubation step Intermediate XVI (96.4 kg, 354.7117 mol) and dichloromethane (321 kg) were added, and the resulting suspension was stirred at 20° C. for 1 h. After addition of seeding crystals of Intermediate XVII 50.2 kg tert-butylhydroperoxide solution (70% in water) was added slowly in three portions. The reaction mixture was stirred until reaction completion. The precipitate was isolated by filtration, washed with isopropylacetate and dried at 40° C. in the filter dryer to afford Intermediate XVII as orange solid in 94.2% yield. The isolated product contained 5.8-6.0% water.

¹H NMR (500 MHZ, DMSO-d₆) δ=8.65 (s, 1H), 4.91 (t, J=5.8 Hz, 1H), 3.74-3.66 (m, 2H), 3.57 (td, J=7.7, 17.9 Hz, 1H), 3.41-3.27 (m, 1H), 3.13 (ddd, J=1.8, 8.2, 17.7 Hz, 1H), 3.07-2.95 (m, 1H), 2.31-2.12 (m, 4H), 1.83-1.70 (m, 2H).

LCMS (ESI pos) for C₁₁H₁₄ClN₃O₂S, (M+H)+ calcd. 288.1, measd. 288.1

The new preincubation step of S-(−)-Binaphthol in dichloromethane with titanium-(IV)-isopropoxide for about one hour prior to the addition of Intermediate XVI makes it possible to use a reduced amount of the expensive catalyst titanium-(IV)-isopropoxide. Here Intermediate XVI was used more than 90-fold (preferably more than 95-fold, in particular more than 97-fold) in excess compared to titanium-(IV)-isopropoxide (whereas the in U.S. Pat. No. 8,609,670 the corresponding Compound VIII has been used only 20-fold in excess compared to titanium-(IV)-isopropoxide). The preincubation step therefore makes the stereoselective oxidation step yielding in Intermediate XVII more cost-efficient.

The below-mentioned experiments show the correlation between the triethylammonium salt content of Intermediate XVI and the enantiomeric impurity ent-XVII

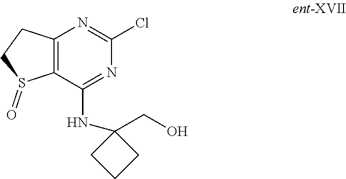 [SMILES: O=[S@]1CCc2nc(Cl)nc(NC3(CO)CCC3)c21]

and the titanium-content of Intermediate XVII. At levels below 0.8% TEA*HCl in Intermediate XVI, the enantiomeric impurity ent-XVII is below detection limit in Intermediate XVII (see example 1 and 2) as well as low levels of titanium have been observed (see example 1). In absence of the salt TEA*HCl (content TEA*HCL salt T<0.5%) a significantly low titanium content of 27 ppm is determined in the isolated product. At higher triethylammonium hydrochloride contents in Intermediate XVI, higher titanium contents are observed in the isolated Intermediate XVII (see examples 3-5). The described triethylammonium hydrochloride free quality of Intermediate XVI also allows the isolation of Intermediate XVII in very high enantiomeric excess of ee>99.88%. In comparison to similar examples in the literature [a) S. Uemura et al., Tetrahedron Lett. 1992, 33, 5391, b) J. Gao et al., Tetrahedron Lett. 2007, 48, 8453; c) N. Komatsu et al., J. Org. Chem 1993, 58, 4529], which describe enantioselective oxidations of thioethers to sulfoxides using Uemura catalyst [S. Uemura et al., Tetrahedron Lett. 1992, 33, 5391], the enantiomeric purity is higher according to the enantioselective oxidation of the invention.

| | Intermediate | Intermediate | | | |
|---|---|---|---|---|---|
| | XVI | XVII | | | |
| | TEA*HCl | ent-XVII | Ti content | Scale | Yield |
| Example | content [%] | content [%] | [mg/kg] | [g] | [%] |
| 1 | <0.5 | <0.06 | 27 | 30.0 | 88.9 |
| 2 | 0.8 | <0.06 | 1080 | 30.0 | 90.0 |
| 3 | 3.4 | 0.39 | 611 | 30.0 | 92.3 |
| 4 | 5.7 | 1.5 | 1293 | 30.0 | 89.7 |
| 5 | 10.6 | 1.7 | 1128 | 30.0 | 86.9 |

### 3.8 Synthesis of Intermediate XVIII

In contrast to the manufacturing process of Example 2 as described in U.S. Pat. No. 8,609,670B, the manufacturing process of the PDE4B-inhibitor of formula XX according to the invention includes an additional recrystallization step after the enantioselective oxidation step.

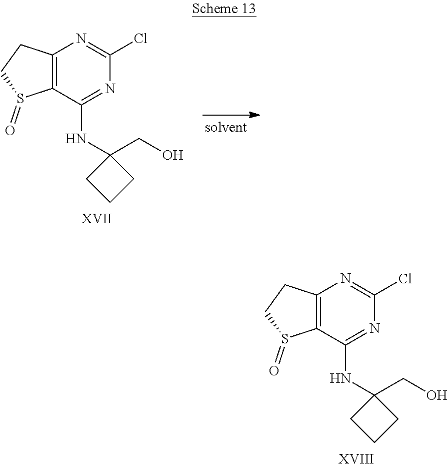

### 3.8.1 Synthesis of Intermediate XVIII: Variant A

In a vessel 25 kg (1 eq) of Intermediate XVII were suspended in 8.0 VP of 1,4-dioxane. The mixture was heated to 80-90° C. and stirred for 1 hour. The solution was filtered and at 85° C. concentrated to 3.0 volume parts. At 70-80° C. seeding crystals of Intermediate XVIII (0.1 m %) were added to the remaining solution and stirred for 30 min. The suspension was slowly cooled to 22° C. and stirred for 2 h. The precipitate was isolated by filtration and the filter cake was washed with 2.0 VP of acetonitrile. The product was dried in a filter dryer at 60° C. to afford Intermediate XVIII in 63.2% yield.

¹H NMR (500 MHZ, DMSO-d₆) δ=8.65 (s, 1H), 4.91 (t, J=5.8 Hz, 1H), 3.74-3.66 (m, 2H), 3.57 (td, J=7.7, 17.9 Hz, 1H), 3.41-3.27 (m, 1H), 3.13 (ddd, J=1.8, 8.2, 17.7 Hz, 1H), 3.07-2.95 (m, 1H), 2.31-2.12 (m, 4H), 1.83-1.70 (m, 2H).

LCMS (ESI pos) for C₁₁H₁₄ClN₃O₂S, (M+H)+ calcd. 288.1, measd. 288.1

The recrystallization procedure of Intermediate XVII according to variant A controls the titanium content in the isolated material as shown in the below table. In 1,4-dioxane, the titanium-impurities are insoluble and are separated by filtration. The recrystallization process according to variant A therefore ensures titanium levels ≤30 ppm in the sulfoxide Intermediate XVIII (see Ti contents of Intermediates XVIII of 23 mg/kg and of 30 mg/kg for examples 1 and 2). Additionally, the examples 1 and 2 show the full purge of ent-XVII from levels of 0.85% and 1.17% below 0.06%, allowing the isolate XVIII in optical pure quality.

| | | | | | | | Loss on |
|---|---|---|---|---|---|---|---|
| | | | | | | | Drying |
| | ent-XVII content [%] in | | Ti content [mg/kg] in | | | | (LoD) [%] |
| | Intermediate | Intermediate | Intermediate | Intermediate | Scale | Yield | Intermediate |
| Example | XVII | XVIII | XVII | XVIII | [kg] | [%] | XVIII |
| 1 | 0.85 | <0.06 | 958 | 23 | 31.5 | 56.2** | 0.03 |
| 2 | 1.17* | <0.06 | 2568* | 30 | 25.0 | 63.2 | 0.06 |

*calculated content (two batches of Intermediate XVII were used).
**lower yield due to tailing effect in the pilot plant isolation equipment (first batch
production).

In comparison to the process as described in U.S. Pat. No. 8,609,670B, the additional recrystallization step after the enantioselective oxidation step according to variant A yielding Intermediate XVIII also improves the quality of the sulfoxide XVII critically. Hereby, Intermediate XVIII is obtained as a water-free form. The water-free material is long-term stable (>12 months) regarding hydrolysis side-reactions, which can occur at two positions of Intermediate XVII by forming the impurities IMP-A and IMP-B (see Scheme 14).

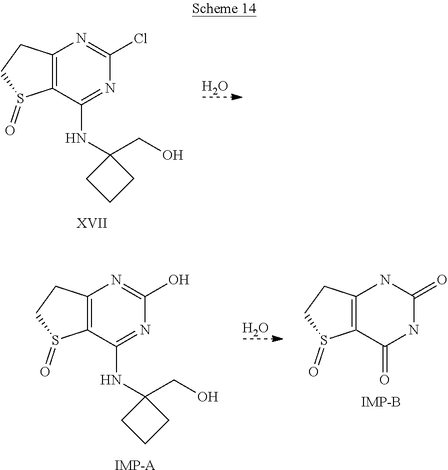

Degradation of XVII by hydrolysis.

The table below shows stress test-experiments of the hydrolysis reactions of Intermediate XVII. It is shown, that hydrolysis toward IMP-A occurs at 60° C. and 90° C. (examples 1 and 2). The hydrolysis reaction itself is promoted by titanium residues, as example 2 with 671 mg/kg titanium content leads to higher impurity levels of IMP-A compared to example 1, which has a lower titanium content (<251 mg/kg).

| | IMP-A content [%] in | | | IMP-B content [%] in | | | |
|---|---|---|---|---|---|---|---|
| | | XVII after | XVII after | | XVII after | XVII after | Ti |
| | XVII at | 1 week at | 1 week at | XVII at | 1 week at | 1 week at | content |
| Example | t = 0 | 60° C. | 90° C. | t = 0 | 60° C. | 90° C. | [mg/kg] |
| 1 | 0.10 | 0.13 | 0.16 | <0.05 | 0.05 | 0.34 | <251 |
| 2 | <0.05 | 0.19 | 0.32 | <0.05 | <0.05 | 0.54 | 671 |

### 3.8.2 Synthesis of Intermediate XVIII: Variant B

In a vessel under inert atmosphere 88.0 kg (1.0 eq) of Intermediate XVII was suspended in 345.4 kg (5.0 VP) acetonitrile and 176.0 kg (2.0 VP) water. The mixture was heated to 70-80° C. and stirred for 1 hour. The solution was filtered, and the filter was washed with 138.6 kg acetonitrile (2.0 VP). After that the filtrate was concentrated at 75° C. to 3 volume parts. 528.0 kg water (6.0 VP) was added, and the mixture was heated to 70-80° C. After cooling to ca. 60° C. seeding crystals of Intermediate XVIII (0.1 m %) were added to the solution and the solution was stirred for 15 min. The suspension was slowly cooled to 10° C. and stirred for 1 hour. The precipitate was isolated by filtration and the filter cake was washed with 280.0 kg (4.0 VP) isopropyl acetate. The product was dried in the filter dryer at 40° C. to afford Intermediate XVIII in 86.3% yield.

¹H NMR (500 MHZ, DMSO-d₆) δ=8.65 (s, 1H), 4.91 (t, J=5.8 Hz, 1H), 3.74-3.66 (m, 2H), 3.57 (td, J=7.7, 17.9 Hz, 1H), 3.41-3.27 (m, 1H), 3.13 (ddd, J=1.8, 8.2, 17.7 Hz, 1H), 3.07-2.95 (m, 1H), 2.31-2.12 (m, 4H), 1.83-1.70 (m, 2H).

LCMS (ESI pos) for C₁₁H₁₄ClN₃O₂S, (M+H)+ calcd. 288.1, measd. 288.1

As demonstrated by examples 1 and 2 in the below table, applying the additional recrystallization step of Intermediate XVII according to variant B significantly removed titanium to levels below 20 ppm in Intermediate XVIII (see table below: Ti-contents of Intermediate XVIII of <11 mg/kg and of 18 mg/kg). In addition, ent-XVII could be purged by recrystallization from 0.59% to below the detection limit of <0.06% in Example 2. It is noteworthy, that recrystallization variant B yields Intermediate XVIII with water contents between 5.0 and 6.0% (unlike variant A and C).

| | ent-XVII content [%] in | | Ti content [mg/kg] in | | | |
|---|---|---|---|---|---|---|
| | Intermediate | Intermediate | Intermediate | Intermediate | Scale | Yield |
| Example | XVII | XVIII | XVII | XVIII | [kg] | [%] |
| 1 | <0.06 | <0.06 | 927 | <11 | 88.0 | 86.3 |
| 2 | 0.59 | <0.06 | 1184 | 18 | 60.0 | 86.2 |

### 3.8.3 Synthesis of Intermediate XVIII: Variant C

50.0 g (1.0 eq) of Intermediate XVII was dissolved in tetrahydrofuran (7.0 VP). 10 m % active carbon was added, and the mixture was heated to reflux and allowed to stir for 30 min. The mixture was filtered and concentrated to 2.5 volume parts. At 55° C. seeding crystals of Intermediate XVIII (0.1 m %) were added to the remaining solution and the solution was stirred for 30 min. 2.5 VP isopropyl acetate were added and the solution was stirred for 30 min, then the mixture was cooled to 10° C. The precipitate was isolated by filtration and the filter cake was washed with acetonitrile (2.0 VP). The product was dried at 40° C. in vacuo to afford Intermediate XVIII in 85.8% yield.

¹H NMR (500 MHZ, DMSO-d₆) δ=8.65 (s, 1H), 4.91 (t, J=5.8 Hz, 1H), 3.74-3.66 (m, 2H), 3.57 (td, J=7.7, 17.9 Hz, 1H), 3.41-3.27 (m, 1H), 3.13 (ddd, J=1.8, 8.2, 17.7 Hz, 1H), 3.07-2.95 (m, 1H), 2.31-2.12 (m, 4H), 1.83-1.70 (m, 2H).

LCMS (ESI pos) for C₁₁H₁₄ClN₃O₂S, (M+H)+ calcd. 288.1, measd. 288.1

As demonstrated by examples 1 and 2 in the below table, applying the additional recrystallization step of Intermediate XVII according to variant C significantly removed titanium to levels below 30 ppm in Intermediate XVIII (see below table: Ti-content of Intermediate XVIII is 26 mg/kg or 12 mg/kg). In addition, the unwanted enantiomer ent-XVII could be purged by recrystallization from 0.85% to below the detection limit of <0.06%. It is noteworthy, that the recrystallization step according to variant C yields Intermediate XVIII in a water free form (identical to variant A).

| | ent-XVII content [%] in | | Ti content [mg/kg] in | | | |
|---|---|---|---|---|---|---|
| | Intermediate | Intermediate | Intermediate | Intermediate | Scale | Yield |
| Example | XVII | XVIII | XVII | XVIII | [g] | [%] |
| 1 | <0.06 | <0.06 | 853 | 26 | 50 g | 85.8 |
| 2 | 0.85 | <0.06 | 958 | 12 | 50 g | 78.8 |

### 3.9 Synthesis of Intermediate XIX

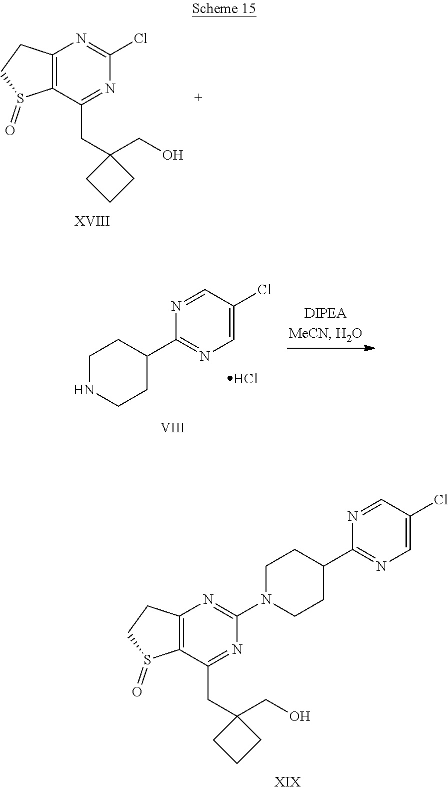

30.7 kg Intermediate XVIII and 23.7 kg Intermediate VIII were charged in a vessel under inert atmosphere and suspended in an acetonitrile (96 kg)/water (184 kg) mixture. The suspension was heated to 70° C. and 27.6 kg of N,N-diisopropylethylamine (DIPEA) were added. After that the mixture was heated to reflux and stirred for approx. 1 hour. After reaction completion the suspension was cooled to 20° C. and stirred for at least 1 hour. The product was isolated by centrifugation, washed with water, and dried at 60° C. in an agitated dryer to afford Intermediate XIX in 86.1% yield.

¹H NMR (500 MHZ, DMSO-d₆) δ=8.86 (s, 1H), 7.38 (s, 1H), 4.85 (t, J=5.7 Hz, 1H), 4.70 (br d, J=11.5 Hz, 2H), 3.72 (br dd, J=2.8, 5.3 Hz, 2H), 3.48-3.36 (m, 1H), 3.26-3.14 (m, 2H), 3.12-3.02 (m, 2H), 2.93 (ddd, J=1.5, 8.2, 17.1 Hz, 1H), 2.86 (ddd, J=1.5, 7.3, 13.5 Hz, 1H), 2.40-2.24 (m, 2H), 2.22-2.12 (m, 2H), 1.96 (br d, J=12.7 Hz, 2H), 1.86-1.70 (m, 2H), 1.70-1.57 (m, 2H).

LCMS (ESI pos) for C₂₀H₂₅ClN₆O₂S, (M+H)+ calcd. 449.2, measd. 449.2

In comparison to the manufacturing process of Example 2 as described in U.S. Pat. No. 8,609,670B, this manufacturing step to yield Intermediate XIX has been amended as follows:
The amount of N,N-diisopropylethylamine (DIPEA) could be reduced from 2.5 eq. to 2.1 eq., allowing less waste of the ecotoxic base DIPEA.
The use of acetonitrile and water as solvents allows a fast conversion time of less than 60 min instead of 180 min. The use of the potential cancerogenic solvent tetrahydrofuran (THF) and acetone as washing medium could be avoided. It was observed that acetone can be substituted by the “green solvent” water.

### 3.10 Synthesis of the PDE4B-Inhibitor of Formula XX

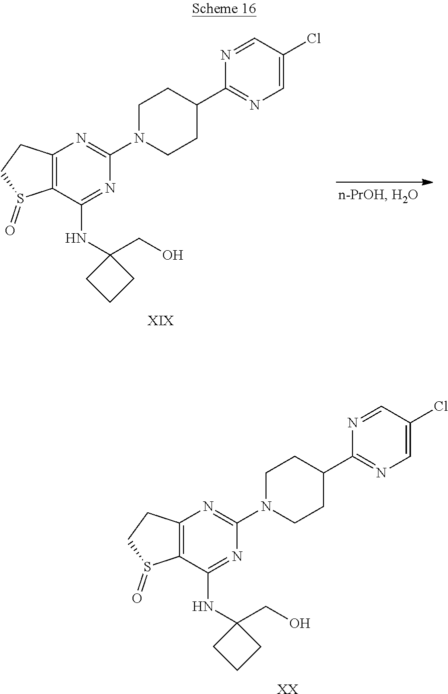

Intermediate XIX (42.9 kg) was charged in a vessel under inert atmosphere and dissolved in a mixture of n-propanol (189 kg; 5.0 VP) and water (54 kg; 1.25 VP) under reflux conditions. The solution was then kept at 80° C. and filtered. The filter was washed with n-propanol (69 kg, 2.0 VP). The solution was again heated to reflux and afterwards cooled to 70° C. Seeding crystals of the Compound of formula XX (0.2 kg; 0.5 m %) were added and the mixture was stirred for 1 hour (crystalline form A was formed). The mixture was then cooled to 20° C. within 90 min. After stirring for 30 min the mixture was heated again to 40° C. within 30 min until crystalline form B of the Compound of formula XX was formed within 4 hours. The mixture was then cooled to room temperature within 50 min and n-propanol (326 kg; 10.0 VP) was added. After that the mixture was cooled to 0-5° C. and the product was isolated after 1 hour by centrifugation. After washing with n-propanol (137 kg; 4.0 VP) the product was dried at 60° C. in an agitated dryer to afford Compound XX in 85.3% yield.

¹H NMR (500 MHZ, DMSO-d₆) δ=8.86 (s, 1H), 7.38 (s, 1H), 4.85 (t, J=5.7 Hz, 1H), 4.70 (br d, J=11.5 Hz, 2H), 3.72 (br dd, J=2.8, 5.3 Hz, 2H), 3.48-3.36 (m, 1H), 3.26-3.14 (m, 2H), 3.12-3.02 (m, 2H), 2.93 (ddd, J=1.5, 8.2, 17.1 Hz, 1H), 2.86 (ddd, J=1.5, 7.3, 13.5 Hz, 1H), 2.40-2.24 (m, 2H), 2.22-2.12 (m, 2H), 1.96 (br d, J=12.7 Hz, 2H), 1.86-1.70 (m, 2H), 1.70-1.57 (m, 2H).

LCMS (ESI pos) for C₂₀H₂₅ClN₆O₂S, (M+H)+ calcd. 449.2, measd. 449.2

In the light of the phase-diagram of the PDE4B-inhibitor of formula XX, an optimized crystallization process of the Compound of formula XX in the desired crystalline Form B (thermodynamically most stable Form) has been developed. The optimizations compared to the corresponding crystallization processes as described in U.S. Pat. No. 8,609,670B include the following additional steps:
a) an additional clear filtration of the solution before cooling
b) after the cooling to 20° C. the solution is reheated to 40° C., since at 40° C. the conversion of Form A (which crystallizes first, undesired crystalline Form) to Form B (which is the desired crystalline Form, since it is the thermodynamically most stable crystalline form) is completed more rapidly (in at least 4 hours)
c) after cooling from 40° C. to room temperature the solution is significantly diluted with n-propanol (10.0 volume parts) and further cooled to 0-5° C.

This crystallization process of the invention which was optimized with the above-mentioned addition process steps (compared to the crystallization process as described in U.S. Pat. No. 8,609,670B) offers a better control of the complete und fast conversion and isolation of the crystalline form B of the Compound of formula XX.

In particular it could be observed, that the reheating step b) to 40° C. indeed minimizes the time of complete conversion of the initially formed undesired crystalline Form A of the Compound of formula XX to the desired, thermodynamically most stable crystalline Form B of the Compound of formula XX to at least 4 hours.

Furthermore, it could be observed that the late dilution with n-propanol in step c) decreases water activity of the solution and that therefore Compound of formula XX is isolated as crystalline Form B (the desired thermodynamically most stable crystalline Form of the Compound of formula XX) and is not transformed into crystalline Form C (the dihydrate Form that is formed at higher water activities, e.g. at water activity greater than 0.87 at ≥20° C.).

### 3.11 X-Ray Powder Diffraction (XRPD) Analysis of of the Compound of Formula XX as Manufactured by the Processes According to the Invention (Anhydrous Crystalline Form B)

The Compound of formula XX as manufactured as described above has been analyzed by X-ray powder diffraction (XRPD) with the following conditions and instrumental setup:
Instrument: D2 Phaser 2nd Gen Bruker; CuKα 2=1.5406 Å
Measurement conditions: 30 kV/10 mA; 3°-40° 2Theta; Reflexion
Increment=0.02° 2Θ/Time/Step=0.3 sec
Sample preparation: ground by mortar/pistle
Specimen Holder Airtight SMPLHLDR/Si SINGL CRST cav 6 XSC A26-B65 (KAPTON® Film 7.5μ)

The below-listed observable XRPD-peaks of this Compound of formula XX as manufactured as described above shows clearly that this Compound of formula XX as manufactured according to the above-mentioned processes of the invention is in the crystalline anhydrous Form B:

| 2-Theta | d(Å) | I/Io |
|---|---|---|
| 4.78 | 18.47 | 46 |
| 9.78 | 9.04 | 25 |
| 14.56 | 6.08 | 5 |
| 15.14 | 5.85 | 17 |
| 16.96 | 5.22 | 43 |
| 17.48 | 5.07 | 14 |
| 19.18 | 4.62 | 100 |
| 19.74 | 4.49 | 41 |
| 20.80 | 4.27 | 38 |
| 21.30 | 4.17 | 71 |
| 21.72 | 4.09 | 28 |
| 23.82 | 3.73 | 50 |
| 24.28 | 3.66 | 55 |
| 24.58 | 3.62 | 35 |
| 25.53 | 3.49 | 4 |
| 26.64 | 3.34 | 21 |
| 27.12 | 3.29 | 2 |
| 27.61 | 3.23 | 13 |
| 27.90 | 3.20 | 31 |
| 28.48 | 3.13 | 8 |
| 28.78 | 3.10 | 18 |
| 29.74 | 3.00 | 8 |
| 30.92 | 2.89 | 18 |
| 31.75 | 2.82 | 6 |
| 32.04 | 2.79 | 10 |
| 32.78 | 2.73 | 2 |
| 34.55 | 2.59 | 8. |

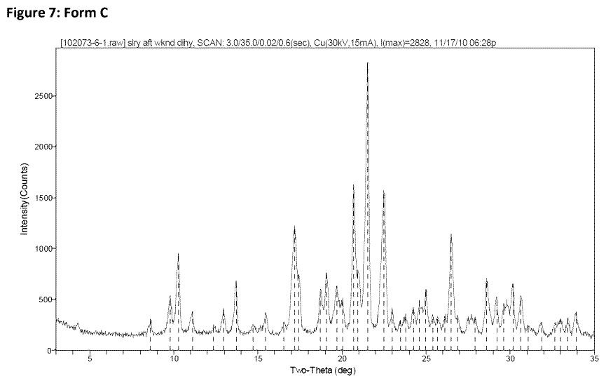

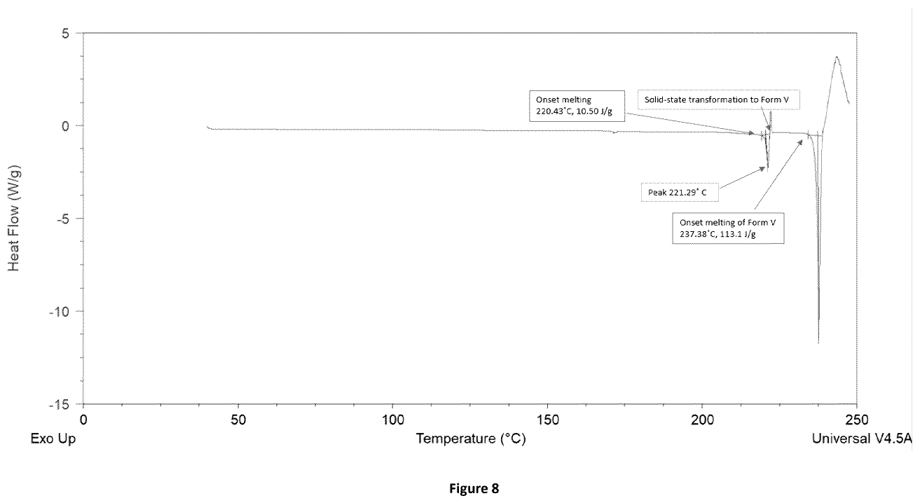

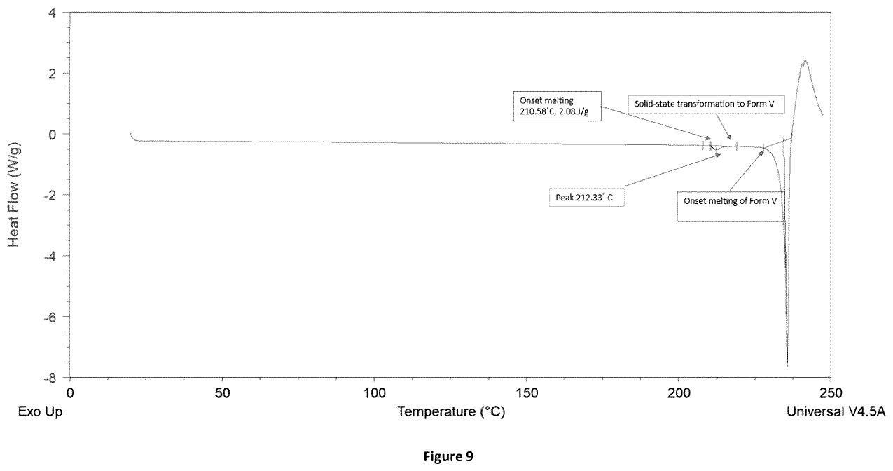

The XRPD-diagram of the Compound of formula XX as manufactured as described above, with the observable peaks as disclosed above is shown in FIG. 6.

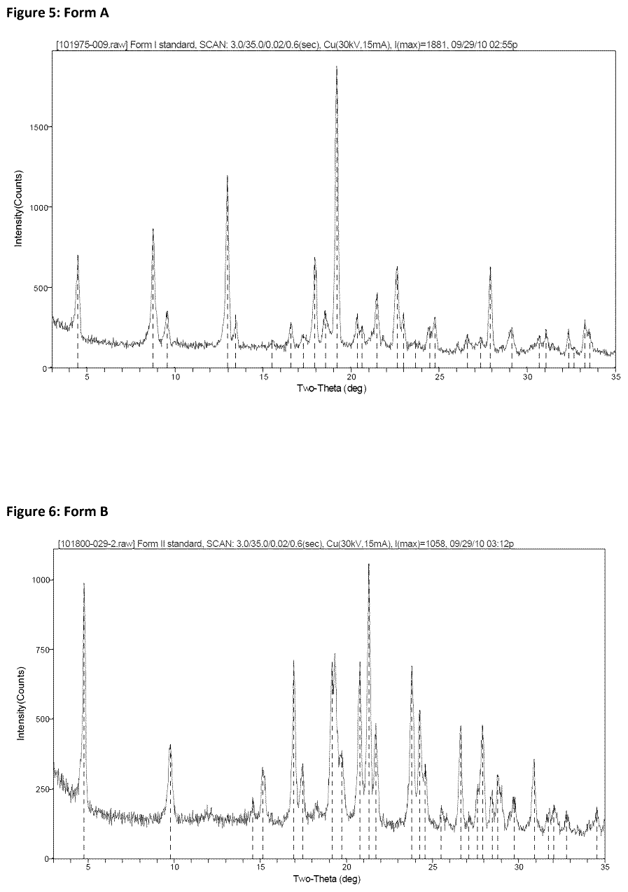

FIGS. 5 and 7 show corresponding XRPD-diagrams of crystalline anhydrous Form A and of crystalline dihydrate form C of the compound with formula

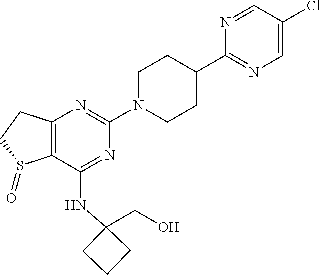 [SMILES: O=[S@@]1CCc2nc(N3CCC(c4ncc(Cl)cn4)CC3)nc(NC3(CO)CCC3)c21]

respectively which have been manufactured according to the processes as described in U.S. Pat. No. 8,609,670B. For the XRPD-analysis of crystalline Forms A and C (as shown in FIGS. 5 and 7, respectively) the same experimental setup and the same conditions as described above had been used.

The XRPD-diagram shown in FIG. 5 of the crystalline Form A of the compound of formula

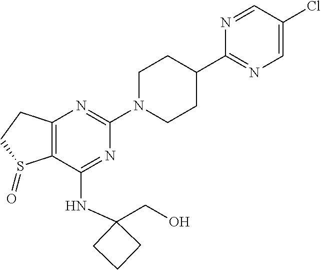 [SMILES: O=[S@@]1CCc2nc(N3CCC(c4ncc(Cl)cn4)CC3)nc(NC3(CO)CCC3)c21]

shows the following observable peaks:

| 2-Theta | d(Å) | I/Io |
|---|---|---|
| 4.48 | 19.70 | 27 |
| 8.76 | 10.09 | 46 |
| 9.54 | 9.26 | 11 |
| 12.98 | 6.82 | 69 |
| 13.44 | 6.58 | 9 |
| 15.50 | 5.71 | 2 |
| 16.56 | 5.35 | 8 |
| 17.94 | 4.94 | 35 |
| 18.54 | 4.78 | 20 |
| 19.18 | 4.62 | 100 |
| 20.36 | 4.36 | 15 |
| 20.64 | 4.30 | 10 |
| 21.48 | 4.13 | 23 |
| 22.62 | 3.93 | 38 |
| 22.98 | 3.87 | 15 |
| 23.65 | 3.76 | 4 |
| 24.46 | 3.64 | 15 |
| 24.76 | 3.59 | 21 |
| 26.61 | 3.35 | 5 |
| 27.34 | 3.26 | 13 |
| 27.92 | 3.19 | 28 |
| 29.14 | 3.06 | 15 |
| 30.68 | 2.91 | 11 |
| 31.05 | 2.88 | 17 |
| 32.34 | 2.77 | 8 |
| 32.65 | 2.74 | 3 |
| 33.28 | 2.69 | 20 |
| 33.54 | 2.67 | 17 |

The XRPD-diagram shown in FIG. 7 of the crystalline Form C of the compound of formula

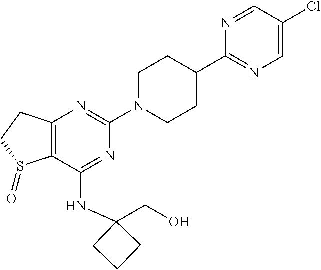 [SMILES: O=[S@@]1CCc2nc(N3CCC(c4ncc(Cl)cn4)CC3)nc(NC3(CO)CCC3)c21]

shows the following observable peaks:

| 2-Theta | d(Å) | I/Io |
|---|---|---|
| 8.60 | 10.27 | 4 |
| 9.78 | 9.04 | 15 |
| 10.28 | 8.60 | 28 |
| 11.10 | 7.97 | 6 |
| 12.96 | 6.83 | 8 |
| 13.72 | 6.45 | 16 |
| 14.72 | 6.01 | 5 |
| 15.46 | 5.73 | 9 |
| 17.20 | 5.15 | 70 |
| 18.72 | 4.74 | 21 |
| 19.10 | 4.64 | 29 |
| 19.70 | 4.50 | 33 |
| 20.04 | 4.43 | 26 |
| 20.70 | 4.29 | 75 |
| 21.54 | 4.12 | 100 |
| 22.48 | 3.95 | 61 |
| 23.00 | 3.86 | 5 |
| 23.78 | 3.74 | 5 |
| 24.26 | 3.67 | 7 |
| 24.62 | 3.61 | 15 |
| 24.98 | 3.56 | 19 |
| 26.50 | 3.36 | 41 |
| 27.92 | 3.19 | 8 |
| 28.62 | 3.12 | 20 |
| 29.21 | 3.05 | 9 |
| 29.64 | 3.01 | 15 |
| 30.18 | 2.96 | 19 |
| 30.66 | 2.91 | 16 |
| 31.88 | 2.80 | 4 |
| 33.00 | 2.71 | 12 |
| 33.94 | 2.64 | 9 |

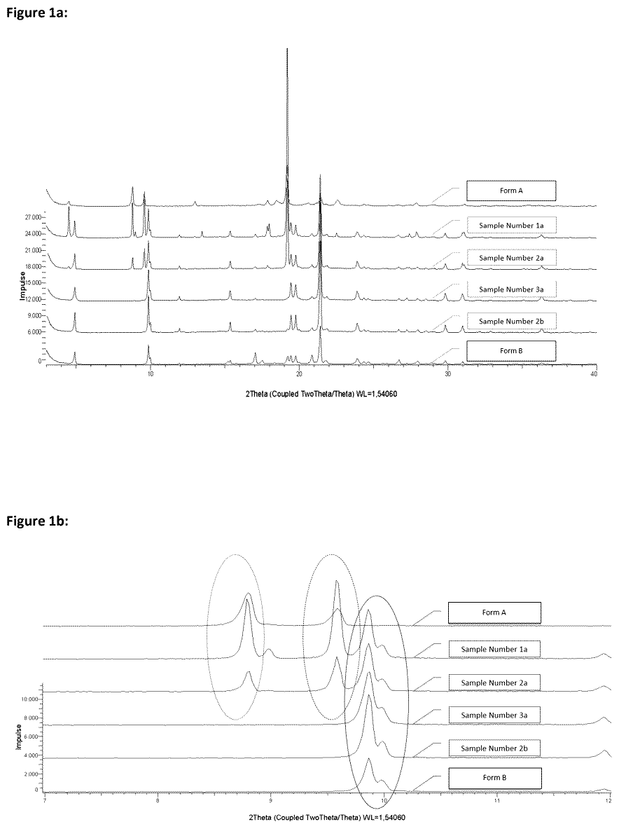

Consequently, and as shown by the experiments in Chapter 4.2 the Compound of formula XX as manufactured according to the invention is therefore a crystalline anhydrous Form B of the Compound of formula XX having a powder X-ray diffraction pattern comprising peaks at the following 2 Theta values measured using CuKα radiation: 19.18±0.2; 21.30±0.2; 24.28±0.2; 23.82±0.2 and 4.78±0.2, but not comprising a peak at the 2 Theta value of 8.76±0.2 measured using CuKα radiation (as this is an XRPD-peak only appearing e.g. in samples comprising also crystalline Form A (see Chapter 4.2 and FIGS. 1a and 1b).

### 3.12 Differential Scanning Calorimetry of the Compound of Formula XX as Manufactured by the Processes According to the Invention (Anhydrous Crystalline Form B) and of its Crystalline Form A (Anhydrous Crystalline Form A)

The Compound of formula XX as manufactured by the processes according to the invention and also its corresponding “crystalline anhydrous Form A” as manufactured as described in U.S. Pat. No. 8,609,670B have been characterized with a TA Instruments Differential Scanning calorimeter (DSC) as shown in FIGS. 8 and 9, respectively.

Hereby the corresponding samples have been analyzed in an unsealed Aluminium pan under an N₂ flow. The temperature ramp that has been used for the measurement was 10° C./min to 250° C.

The thermal behavior of the Compound of formula XX as manufactured according to the processes of the invention is shown in FIG. 8.

The DSC diagram of the Compound of formula XX as manufactured according to the processes of the invention in FIG. 8 (in its crystalline Form B) shows an endotherm onset at 220.43±2° C. with an enthalpy of 10.5±5 J/g and a peak at 221.29±2° C. The thermal event showed melting and a solid-state phase transformation to the “high temperature Form V” of the Compound of formula XX. The endotherm onset at 237.38±2° C. indicates the melting of the high temperature Form V of the Compound of formula XX.

FIG. 9 shows the results of a corresponding differential scanning calorimetry (DSC) experiment of a sample of the compound of formula

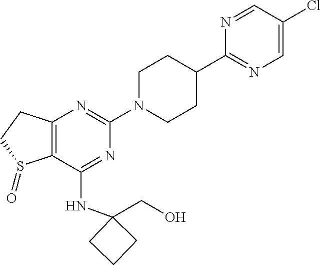 [SMILES: O=[S@@]1CCc2nc(N3CCC(c4ncc(Cl)cn4)CC3)nc(NC3(CO)CCC3)c21]

in its crystalline Form A (as manufactured as described in U.S. Pat. No. 8,609,670B). Crystalline Form A shows a small endotherm at 210.58±2° C. with an enthalpy of 2.08±1 J/g and peak at 212.33±2° C. Thermal events showed solid-state phase transformation to the “high temperature Form V” and the melting of Form V.

### 4. FURTHER EXPERIMENTS CONCERNING THE IDEAL CONDITIONS FOR RECRYSTALLIZATION OF THE COMPOUND OF FORMULA XX IN CRYSTALLINE FORM B

### 4.1 Relevant Crystalline Forms of the Compound of Formula XX in the Recrystallization Process

Crystalline Form B is the thermodynamically most stable form of the PDE4B-inhibitor of formula XX. Few other crystalline forms were observed during the initial and extended solid form screening by using various techniques. Process relevant crystalline forms of the PDE4B-inhibitor of formula XX are crystalline Form A (anhydrous form, kinetically preferred form), crystalline Form B (anhydrous form, thermodynamically most stable form) and crystalline Form C (dihydrate form). Its thermodynamic stability has been confirmed by competitive slurries containing Form A, Form B and Form C. Form B was the predominant form in all solvents and temperature conditions, except for the solvent water (with a water activity of 1) at temperatures between 5 and 25° C. which yielded Form C.

### 4.2 Experiment 1 for the Identification of the Ideal Crystallization Conditions to Yield the Compound of Formula XX in Crystalline Form B

The Intermediate of formula XIX has been manufactured in production scale batches according to the processes as described in Chapters 3.1 to 3.9 of the instant application. Then the Intermediate of formula XIX has been crystallized into the Compound of formula XX in its crystalline Form B according to the procedure as described in Chapter 3.10 of the application involving the following steps:
d1) Intermediate XIX was dissolved in a mixture of 5.0 VP of n-propanol and of 1.25 VP of water under reflux conditions
d2) the solution was kept at 80° C. and filtered
d3) the solution is washed with 2VP of n-propanol
d4) the solution was again heated to reflux and afterwards cooled to 70° C.
d5) seeding crystals of Compound XX were added and the mixture was stirred for at least 1 hour
d6) the mixture was then cooled to 20° C. within at least 90 minutes
d7) after stirring for at least 30 minutes the mixture was then heated again to a temperature of 40° C. within at least 30 minutes to yield crystalline form B of Compound XX within at least 4 hours
d8) the mixture is cooled to room temperature and 10.0 VP of n-propanol was added
d9) the mixture was cooled to 0-5° C. and after at least 1 hour the PDE4B-inhibitor of formula XX in crystalline form B was isolated
d10) after washing with n-propanol the PDE4B-inhibitor of formula XX in crystalline form B was dried.

After step d6) sample 1a has been taken.

After reaching the temperature 40° C. in step d7) sample 2a has been taken.

After stirring for 2 hours at 40° C. in step d7) sample 2b has been taken.

After stirring for 4 hours at 40° C. in step d7) sample 3a has been taken.

Samples 1a, 2a, 2b and 3a have been analyzed by X-ray powder diffraction (XRPD) with the following conditions and instrumental setup:
Instrument: D2 Phaser 2nd Gen Bruker; CuKα 2=1.5406 Å
Measurement conditions: 30 kV/10 mA; 3°−40° 2Theta; Reflexion
Increment=0.02° 2Θ/Time/Step=0.3 sec
Sample preparation: ground by mortar/pistle
Specimen Holder Airtight SMPLHLDR/Si SINGL CRST cav 6 XSC A26-B65 (KAPTON® Film 7.5μ)

As it can been seen from the XRPD-diagrams of samples 1a, 2a, 2b and 3a shown in FIG. 1a and even better from the enlargements of the 2Theta-ranges from 8 to 11 degrees of the XRPD-diagrams of samples 1a, 2a, 2b and 3a shown in FIG. 1b samples numbers 1a (taken at 20° C. internal temperature after step d6)) and 2a (taken directly after reaching 40° C. internal temperature in step d7)) both still contain a mixture of crystalline Form A and B of the Compound of formula XX, whereas sample 2b (taken after stirring at 40° C. for 2 hours in step d7)) and sample 3a (taken after stirring at 40° C. for 4 hours in step d7)) both contain crystalline Form B of the Compound of formula XX only. This shows that the reheating step to 40° C. is crucial to obtain the Compound of formula XX completely in crystalline Form B (and not in a mixture of Forms A and B).

| Sample | Sampling | Crystal Form |
|---|---|---|
| no. | timepoint | (XRPD) |
| 1a | at 20° C. internal | mixture of Form A |
| | temperature | and Form B |
| 2a | after reaching 40° C. | mixture of Form A |
| | internal temperature | and Form B |
| 2b | after stirring for 2 hr at | Form B |
| | 40° C. internal temperature | |
| 3a | after stirring for 4 hr at | Form B |
| | 40° C. internal temperature | |

### 4.3 Conversion into Crystalline Form B of the Compound of Formula XX at Different Temperatures

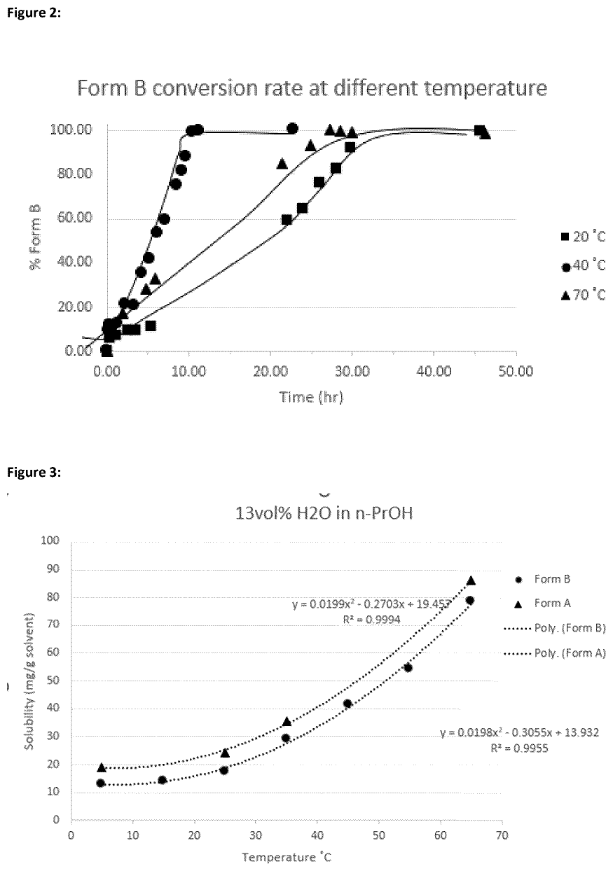

Form A was charged to the reactor as the starting material. 7.0 VP of n-propanol and of 1.25 VP were used along with Raman probe to confirm the rate of conversion by holding the conversion at different temperature. The rate of form conversion from crystalline Form A to crystalline Form B of the PDE4B-inhibitor of formula XX was studied at 20, 40 and 70° C. with 10% seeding with crystalline Form B seeding crystals. The conversion to Form B was determined by Raman spectroscopy as shown in FIG. 2.

FIG. 2 shows that—when the temperature is at 40° C.—the form conversion rate from crystalline Form A to crystalline Form B appears to be the fastest. At 20 or 70° C., the conversion rate was similar, but definitely slower than at 40° C.

The experiments as described in FIG. 2 show that the conversion of crystalline Form A into crystalline Form B of the Compound of formula XX is significantly faster if seeding takes place at a temperature of 40° C., than if seeding takes place at 20° C. or at 70° C. The temperature of 40° C. allows a full conversion of crystalline Form A into crystalline Form B in less than 4 hours, preferably even in 2 hours (see also FIGS. 1a and 1b). In comparison to U.S. Pat. No. 8,609,670B the final crystallization process yielding in the Compound of formula XX in crystalline Form B according to the invention is therefore significantly faster from the timepoint of seeding. The crystallization procedure to yield the thermodynamically most stable crystalline Form B as described in U.S. Pat. No. 8,609,670B needs at least 13.5 hours (on lab scale) from the timepoint of seeding to the timepoint of filtration. However, the crystallization procedure to yield the Compound of formula XX as crystalline Form B (on production scale) as described in Chapter 3.10 reduces the crystallization time down to not more than 9 hours from the timepoint of seeding, which means a significant improvement in the cycling time.

Therefore the final crystallization step to yield crystalline Form B of the PDE4B-inhibitor of formula XX includes the re-heating of the mixture to 40° C. within 30 min until crystalline Form B of the compound of formula XX is formed within ≤4 hours after the seeding step which leads to a fast and complete form conversion of crystalline Form A to crystalline Form B (which is the thermodynamically most stable and therefore preferred crystalline form of the PDE4B-inhibitor of formula XX).

### 4.4 Solubility of the Crystalline Forms A and B of the PDE4B-Inhibitor of Formula XX in n-Propanol and Water

Separate saturation solutions of Form A and Form B in 13 vol % water in n-propanol have been prepared at different temperatures (20° C., 40° C. and 70° C.). The saturated solutions have been filtered through a 2 μm PTFE filter. The filtrates have been diluted with methanol and were then analyzed by HPLC against a reference standard. At each temperature, the solubility of crystalline Forms A and B of the Compound of formula XX in 13 vol % water in n-propanol were measured. FIG. 3 shows the solubility curve of both, crystalline Form A and crystalline Form B of the PDE4B-inhibitor of formula XX in 13 vol % H₂O in n-propanol.

### 4.5 Experiment to Analyze the Influence of the Water Activity and Temperature of the Solvent on the Conversion Rate into Crystalline Form B of the Compound of Formula XX

Water activity a_{w} is calculated from the ratio of solvent to water. The water activity a_{w} is a tool to determine the equilibrium for water distribution between different phases.

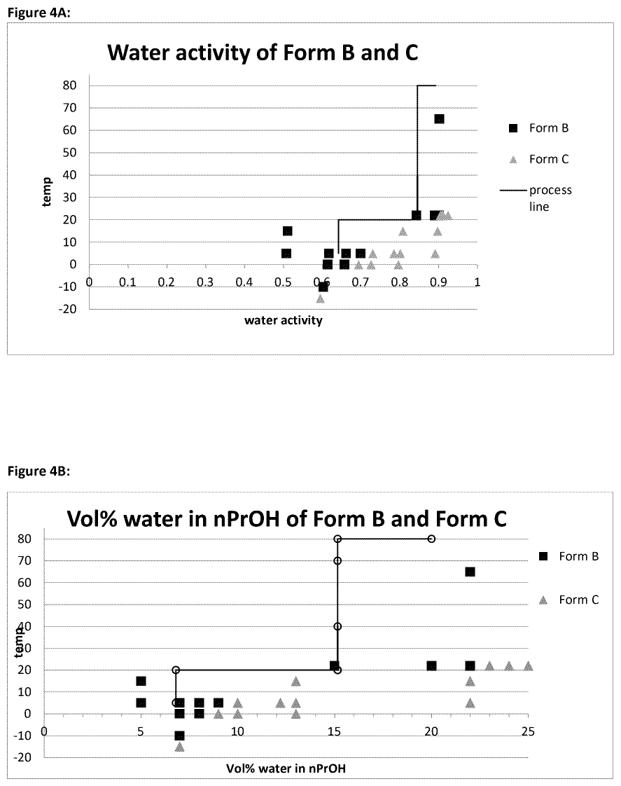

FIG. 4A describes an analysis of the influence of the water activity of the n-propanol/water mixture on the one hand and of the process temperature on the other hand on crystalline form conversions of the Compound of formula XX, whereas the black line (the “process line”) represents the optimized crystallization process to yield the Compound of formula XX in its crystalline Form B only. The additional late dilution step d8) with 10.0 VP of n-propanol at 20° C./room temperature (which decreases water activity from about 0.85 to about 0.65) of the optimized crystallization process of the invention leads to the fact that the “process line” in FIG. 4A ends in an area where predominantly Form B of the Compound of formula XX is formed (see black squares), whereas without that late dilution step d8) at 20° C./room temperature the “process line” would have ended in an area where predominantly the unwanted dihydrate Form C of the Compound of formula XX is formed (see grey triangles).

FIG. 4B shows an analysis of the influence of the volume % of water in n-propanol on the one hand and of the process temperature on the other hand on crystalline form conversions of the Compound of formula XX, whereas the black line (“current process”) represents the optimized crystallization process of the invention to yield the Compound of formula XX in its crystalline Form B only. The additional late dilution step d8) with 10.0 VP of n-propanol at 20° C./room temperature (which decreases the volume % of water in n-propanol from 15 Vol % water to about 7 Vol % water) of the optimized crystallization process of the invention leads to the fact that the “current process” line in FIG. 4B ends in an area where predominantly Form B of the Compound of formula XX is formed (see black squares), whereas without that late dilution step d8) at 20° C./room temperature the “process line” would have ended in an area where predominantly the unwanted dihydrate Form C of the Compound of formula XX is formed (see grey triangles).

As can be concluded from FIG. 4A the unwanted dihydrate Form C of the Compound of formula XX could only be experimentally observed at temperatures of ≤20° C. and when water activity was very high (e.g. at water activities of larger than 0.75 and at temperatures of ≤20° C.).

As can be concluded from FIG. 4B the wanted dihydrate Form C of the Compound of formula XX could only be experimentally observed at temperatures of ≤20° C. and when the Vol % of water in n-propanol was high (e.g. at ≥10 Vol % water in n-propanol and at temperatures of ≤ 20° C.).

Consequently, the late dilution step d8) with 10.0 VP n-propanol at 20° C./room temperature shortly before the solution is cooled to 0-5° C. in step d9) of the final optimized crystallization step to generate Compound of formula XX in its crystalline Form B only decreases the water activity of the n-propanol/water-solvent significantly (that means by that late dilution step the volume % of water in n-propanol at isolation is decreased to about ≤7 Vol % water in n-propanol), so that the undesired late transformation of crystalline Form B into crystalline Form C is completely prevented and only the wanted (thermodynamically most stable) Form B is formed.

### 5. FINAL FORMULATION EXAMPLES FOR THE COMPOUND OF FORMULA XX IN ITS CRYSTALLINE FORM B

The Compound of Formula XX can be formulated as shown in Table A:

TABLE A
Examples for formulations containing the Compound of Formula XX:

| | 9 mg | 18 mg | | Reference to |
|---|---|---|---|---|
| Ingredients | [mg/tablet] | [mg/tablet] | Function | Standards |
| Compound of Formula | 9.000 | 18.000 | Active | |
| XX (in its B-Form) | | | Ingredient | |
| Lactose monohydrate | 85.350 | 170.700 | Filler | Ph. Eur. |
| Cellulose, | 25.500 | 51.000 | Filler | Ph. Eur. |
| microcrystalline | | | | |
| Hydroxypropylcellulose | 3.825 | 7.650 | Binder | Ph. Eur. |
| Croscarmellose sodium | 2.550 | 5.100 | Disintegrant | Ph. Eur. |
| Magnesium stearate | 1.275 | 2.550 | Lubricant | Ph. Eur. |
| Water, purified | * | * | Granulation | Ph. Eur. |
| | | | Liquid | |
| Total (tablet core) | 127.50 | 255.00 | — | — |

* removed during processing, does not appear in the final product

The 9 mg Tablet can be used either with TiO₂-containing or with a TiO₂-free film-coating as shown in Table B.

TABLE B
TiO₂-containing or TiO₂-free film coating of the 9 mg tablet core:

| | 9 mg | 9 mg | | |
|---|---|---|---|---|
| | [mg/tablet] | [mg/tablet] | | |
| | TiO₂- | TiO₂- | | Reference to |
| Ingredients | containing | free | Function | Standards |
| Compound of Formula | 9.000 | 9.000 | Active | |
| XX (in its B-Form) | | | Ingredient | |
| Lactose monohydrate | 85.350 | 85.350 | Filler | Ph. Eur. |
| Cellulose, | 25.500 | 25.500 | Filler | Ph. Eur. |
| microcrystalline | | | | |
| Hydroxypropylcellulose | 3.825 | 3.825 | Binder | Ph. Eur. |
| Croscarmellose sodium | 2.550 | 2.550 | Disintegrant | Ph. Eur. |
| Magnesium stearate | 1.275 | 1.275 | Lubricant | Ph. Eur. |
| Water, purified | * | * | Granulation | Ph. Eur. |
| | | | Liquid | |
| Core mass | 127.50 | 127.50 | — | — |

Film Coat

| | | | | |
|---|---|---|---|---|
| Ingredient | | | | |
| Hypromellose 2910 | 1.400 | 2.450 | Film-forming | Ph. Eur. |
| | | | agent | |
| Mannitol | 0.800 | 1.400 | Plasticizer | Ph. Eur. |
| Macrogol 6000 | 0.480 | — | Plasticizer | Ph. Eur. |
| Macrogol 8000 | — | 0.840 | Plasticizer | Ph. Eur. |
| Talc | 0.720 | 2.303 | Anti-adherent | Ph. Eur. |
| Titanium dioxide | 0.590 | — | Pigment | Ph. Eur., E171 |
| Iron oxide, yellow | 0.010 | 0.007 | Pigment | E172 |
| Total mass of | 4.000 | 7.000 | Film Coating | |
| Film Coat | | | agent | |
| Total mass of Film- | 131.50 | 134.50 | | |
| coated tablet | | | | |

* removed during processing, does not appear in the final product

The 18 mg Tablet can be used either with TiO₂-containing or with a TiO₂-free film-coating as shown in Table C.

TABLE C
TiO₂-containing or TiO₂-free film coating of the 18 mg tablet core:

| | 18 mg | 18 mg | | |
|---|---|---|---|---|
| | [mg/tablet] | [mg/tablet] | | |
| | TiO₂- | TiO₂- | | Reference to |
| Ingredients | containing | free | Function | Standards |
| Compound of Formula | 18.000 | 18.000 | Active | |
| XX (in its B-Form) | | | Ingredient | |
| Lactose monohydrate | 170.700 | 170.700 | Filler | Ph. Eur. |
| Cellulose, | 51.000 | 51.000 | Filler | Ph. Eur. |
| microcrystalline | | | | |
| Hydroxypropylcellulose | 7.650 | 7.650 | Binder | Ph. Eur. |
| Croscarmellose sodium | 5.100 | 5.100 | Disintegrant | Ph. Eur. |
| Magnesium stearate | 2.550 | 2.550 | Lubricant | Ph. Eur. |
| Water, purified | * | * | Granulation | Ph. Eur. |
| | | | Liquid | |
| Core mass | 255.00 | 255.00 | — | — |

Film Coat

| | | | | |
|---|---|---|---|---|
| Ingredient | | | | |
| Hypromellose 2910 | 2.450 | 4.200 | Film-forming | Ph. Eur. |
| | | | agent | |
| Mannitol | 1.400 | 2.400 | Plasticizer | Ph. Eur. |
| Macrogol 6000 | 0.840 | — | Plasticizer | Ph. Eur. |
| Macrogol 8000 | — | 1.440 | Plasticizer | Ph. Eur. |
| Talc | 1.260 | 3.947 | Anti-adherent | Ph. Eur. |
| Titanium dioxide | 1.033 | — | Pigment | Ph. Eur., E171 |
| Iron oxide, red | 0.018 | 0.012 | Pigment | E172 |
| Iron oxide, black | — | 0.001 | Pigment | E172 |
| Total mass | 7.00 | 12.00 | Film Coating | |
| (Film Coat) | | | agent | |
| Total mass | 262.00 | 267.00 | | |
| of tablet | | | | |

* removed during processing, does not appear in the final product

1. Method of manufacturing Intermediate VIII 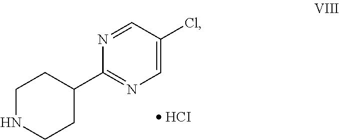
wherein in step a) 5-Chloro-2-indo-pyrimidine and boc-boronic ester V 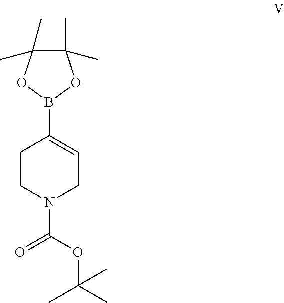 [SMILES: CC(C)(C)OC(=O)N1CC=C(B2OC(C)(C)C(C)(C)O2)CC1]
are reacted in the presence of the catalyst bis(amphos)palladium-II-chloride to yield Intermediate VI 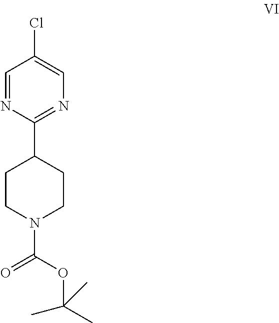 [SMILES: CC(C)(C)OC(=O)N1CCC(c2ncc(Cl)cn2)CC1]
and wherein in step b) Intermediate VI is hydrogenated to yield the non-isolated Intermediate VII 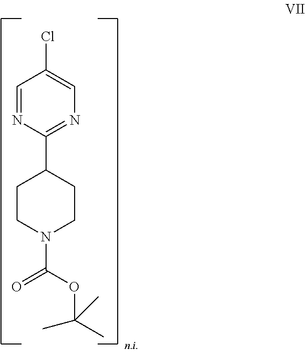
and wherein in step c) the non-isolated Intermediate VII is reacted with hydrochloric acid to yield Intermediate VIII.

2. The method of manufacturing Intermediate VIII according to claim 1, wherein reaction step a) is performed in the presence of tri-potassium-phosphate.

3. The method of manufacturing Intermediate VIII according to claim 1, wherein reaction step a) is performed in the presence of tri-potassium-phosphate, acetonitrile, water and isopropanol.

4. The method of manufacturing Intermediate VIII according to claim 1, wherein reaction step b) is performed in the presence of Raney-Nickel as catalyst.

5. The method of manufacturing Intermediate VIII according to claim 1, wherein reaction step b) is performed in the presence of Raney-Nickel as catalyst in in the presence of methanol as solvent.

6. The method of manufacturing Intermediate VIII according to any of claim 1, wherein reaction step c) is performed in the presence of methanol and methyl-tert-butylether.

7. Method of manufacturing Intermediate XV 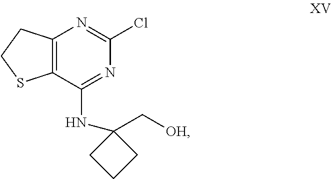 [SMILES: OCC1(Nc2nc(Cl)nc3c2SCC3)CCC1]
wherein 2,4-Dichloro-thienopyrimidine and 1-aminocyclobutyl-methanol hydrochloride are reacted with triethylamine in N-methyl-pyrrolidone at 80° C.

8. The method of manufacturing Intermediate XV according to claim 7, wherein 2,4-Dichloro-thienopyrimidine and 1-aminocyclobutyl-methanol hydrochloride are reacted with triethylamine in N-methyl-pyrrolidone at 80° C. for 6 hours.

9. The method of manufacturing Intermediate XV according to claim 7, wherein 2,4-Dichloro-thienopyrimidine and 1-aminocyclobutyl-methanol hydrochloride are reacted with triethylamine in only 1.0 to 2.0 VP of N-methyl-pyrrolidone at 80° C.

10. The method of manufacturing Intermediate XV according to claim 7, wherein 2,4-Dichloro-thienopyrimidine and 1-aminocyclobutyl-methanol hydrochloride are reacted with 3.0 equivalents of triethylamine in 2.0 VP of N-methyl-pyrrolidone at 80° C.

11. Method of manufacturing Intermediate XVI 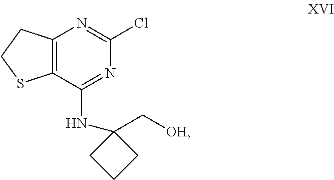 [SMILES: OCC1(Nc2nc(Cl)nc3c2SCC3)CCC1]
which contains ≤0.5% triethylammonium hydrochloride
and which is manufactured
in a first step by the reaction of 2,4-Dichloro-thienopyrimidine and 1-aminocyclobutyl-methanol hydrochloride with triethylamine in N-methyl-pyrrolidone at 80° C. to yield Intermediate XV 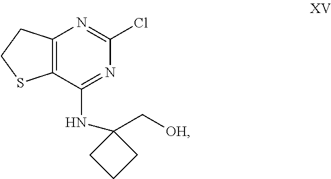 [SMILES: OCC1(Nc2nc(Cl)nc3c2SCC3)CCC1]
whereby this Intermediate XV yielded from the first step is then in a second step subjected to a crystallization from a solvent mixture to yield Intermediate XVI containing ≤0.5% triethylammonium hydrochloride.

12. The method of manufacturing Intermediate XVI which contains ≤0.5% triethylammonium hydrochloride according to claim 11, wherein in the second step
Intermediate XV is suspended in a mixture of n-propanol and water at room temperature
the mixture is then heated to reflux with an internal temperature of 85 to 95° C. and kept at this temperature for at least 30 minutes, then cooled to 60-70° C. internal temperature within at least 30 minutes
after the addition of seeding crystals of Intermediate XVI the reaction mixture is continued stirring at 60-70° C. for at least further 30 minutes
then water is added into the reaction mixture at an internal temperature of 60-70° C. within at least 60 minutes, stirring is continued at 60-70° C. for at least 30 minutes
then the mixture is cooled to an internal temperature of 15-25° C.
then the reaction mixture is filtered, washed with water and dried at a temperature≤75° C.

13. The method of manufacturing Intermediate XVI which contains ≤0.5% triethylammonium hydrochloride according to claim 11, wherein
Intermediate XV is suspended in a mixture of 4 VP of n-propanol and of 7VP of water at room temperature
the mixture is then heated to reflux with an internal temperature of about 89° C. and kept at this temperature for at least 30 minutes, then cooled to 60-70° C. internal temperature within at least 30 minutes
after the addition of seeding crystals of Intermediate XVI the reaction mixture is continued stirring at 60-70° C. for at least further 30 minutes
then water is added into the reaction mixture at an internal temperature of 60-70° C. within at least 60 minutes, stirring is continued at 60-70° C. for at least 30 minutes
then the mixture is cooled to an internal temperature of 20° C.
then the reaction mixture is filtered, washed with water and dried at a temperature≤75° C.

14. Method of manufacturing Intermediate XVI 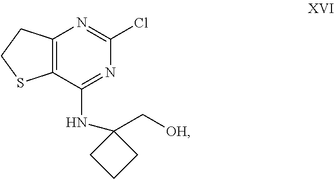 [SMILES: OCC1(Nc2nc(Cl)nc3c2SCC3)CCC1]
which contains ≤0.5% triethylammonium hydrochloride
according to claim 11, whereby Intermediate XV yielded from the first step is then in a second step subjected to a crystallization from a mixture of 2.5 VP of 1,4-dioxane and of 0.5 VP of water to yield Intermediate XVI containing ≤0.5% triethylammonium hydrochloride.

15. The method of manufacturing Intermediate XVI which contains ≤0.5% triethylammonium hydrochloride according to claim 14, wherein in the second step
Intermediate XV is suspended in a mixture of 2.5 VP of 1,4-dioxane and of 0.5 VP of water
the mixture is then heated to 60° C. and kept at this temperature for about 30 minutes
2.0 VP of water is added, seeding crystals of Intermediate XVI are added and the suspension is continued stirring at 60° C. for about 30 minutes
then water is added into the reaction mixture at an internal temperature of 60-70° C. within at least 60 minutes, stirring is continued at 60-70° C. for at least 30 minutes
then the mixture is cooled to an internal temperature of 15-25° C.
then the reaction mixture is filtered, washed with water and dried at a temperature about 80° C.

16. Method of manufacturing a substantially enantiomerically pure Intermediate XVII 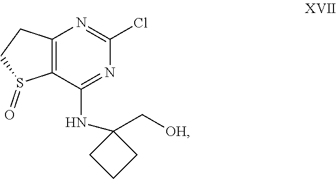 [SMILES: O=[S@@]1CCc2nc(Cl)nc(NC3(CO)CCC3)c21]
whereby this substantially enantiomerically pure Intermediate XVII is manufactured by steps a), b) and c),
whereby
step a) involves the reaction of 2,4-Dichloro-thienopyrimidine and 1-aminocyclobutyl-methanol hydrochloride with triethylamine in N-methyl-pyrrolidone to yield Intermediate XV 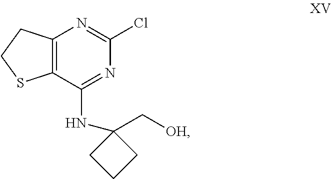 [SMILES: OCC1(Nc2nc(Cl)nc3c2SCC3)CCC1]
step b) involves a crystallization of Intermediate XV yielded in step a) from a mixture of n-propanol and water to yield Intermediate XVI which contains ≤0.5% triethylammonium hydrochloride,
step c) involves the stereoselective oxidation of Intermediate XVI as obtained from step b) with S-(−)-1,1′-Bi-2-naphthol (S-(−)-BINOL), dichloromethane, titanium-(IV)-isopropoxide, water and 70% tert-butylhydroperoxide in water to yield Intermediate XVII.

17. The method of manufacturing the substantially enantiomerically pure Intermediate XVII according to claim 16, wherein Intermediate XVII contains ≤0.06% of the unwanted enantiomer ent-XVII

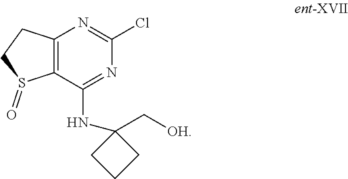 [SMILES: O=[S@]1CCc2nc(Cl)nc(NC3(CO)CCC3)c21]

18. The method of manufacturing the substantially enantiomerically pure Intermediate XVII according to claim 16, wherein Intermediate XVII has an enantiomeric excess of at least 99.88% over the unwanted enantiomer ent-XVII.

19. The method of manufacturing the substantially enantiomerically pure Intermediate XVII according to claim 16, wherein Intermediate XVII contains ≤50 ppm titanium.

20. The method of manufacturing the substantially enantiomerically pure Intermediate XVII according to claim 16, wherein Intermediate XVII contains ≤30 ppm titanium.

21. Method of manufacturing a substantially enantiomerically pure Intermediate XVIII 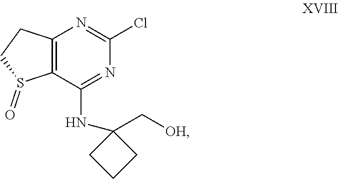 [SMILES: O=[S@@]1CCc2nc(Cl)nc(NC3(CO)CCC3)c21]
which contains ≤0.06% of the unwanted enantiomer ent-XVII 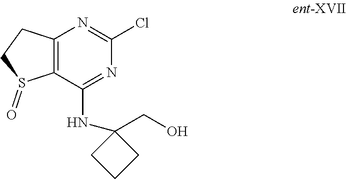 [SMILES: O=[S@]1CCc2nc(Cl)nc(NC3(CO)CCC3)c21]
and which contains ≤30 ppm of titanium,
whereby this substantially enantiomerically pure Intermediate XVII was manufactured by steps a), b), c) and d),
whereby
step a) involves the reaction of 2,4-Dichloro-thienopyrimidine and 1-aminocyclobutyl-methanol hydrochloride with triethylamine in N-methyl-pyrrolidone at 80° C. to yield Intermediate XV 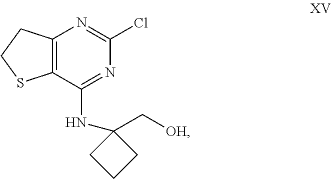 [SMILES: OCC1(Nc2nc(Cl)nc3c2SCC3)CCC1]
step b) involves a crystallization of Intermediate XV yielded in step a) from a mixture of n-propanol and water to yield Intermediate XVI which contains ≤0.5% triethylammonium hydrochloride,
step c) involves the stereoselective oxidation of Intermediate XVI as obtained from step b) with S-(−)-1,1′-Bi-2-naphthol (S-(−)-BINOL), dichloromethane, titanium-(IV)-isopropoxide, water and 70% tert-butylhydroperoxide in water to yield Intermediate XVII,
and step d) involves a recrystallization of Intermediate XVII to yield Intermediate XVIII.

22. The method of manufacturing a substantially enantiomerically pure Intermediate XVIII according to claim 21, wherein this substantially enantiomerically pure Intermediate XVIII has an enantiomeric excess over the unwanted enantiomer ent-XVIII of at least 99.88%.

23. The method of manufacturing Intermediate XVIII according to claim 21, wherein step d) involves a recrystallization of Intermediate XVII from 1,4-dioxane to yield Intermediate XVIII.

24. The method of manufacturing Intermediate XVIII according to claim 23, wherein the recrystallization step d) of Intermediate XVII from 1,4-dioxane to yield Intermediate XVIII involves the following steps:
a1) Intermediate XVII is suspended in 8.0 VP 1,4-dioxane
a2) the mixture is heated to 80-90° C. and stirred until complete dissolution
a3) the solution is filtered and concentrated to 3 volume parts
a4) the concentrated solution is stirred at 70-80° C. for at least 30 minutes
a5) the suspension is slowly cooled to a temperature between 17° C. to 30° C. and stirred
a6) the precipitate is isolated by filtration and the filter cake is washed with acetonitrile or isopropyl acetate
a7) the product is dried to yield Intermediate XVIII.

25. The method of manufacturing Intermediate XVIII according to claim 24, wherein in step a4) seeding crystals of Intermediate XVIII are added to the concentrated solution and the solution is then stirred at 70-80° C. for at least 30 minutes.

26. The method of manufacturing Intermediate XVIII according to claim 24, wherein in step a5) the suspension is slowly cooled to a temperature of 22° C. and wherein in step a6) the filter cake is washed with acetonitrile.

27. The method of manufacturing Intermediate XVIII according to claim 21, wherein step d) involves a recrystallization of Intermediate XVII from a mixture of acetonitrile and water to yield Intermediate XVIII.

28. The method of manufacturing Intermediate XVIII according to claim 27, wherein the recrystallization step d) of Intermediate XVII from a mixture of acetonitrile and water to yield Intermediate XVIII involves the following steps:
b1) Intermediate XVII is suspended in 5.0 volume parts (VP) acetonitrile and 2.0 VP water
b2) the mixture is heated to 70-80° C. and stirred until complete dissolution
b3) the solution is filtered using active carbon, the filter is washed with 2.0 VP acetonitrile and concentrated to 3 volume parts
b4) 6.0 VP water are added and the mixture is heated to 70-80° C.
b5) after cooling to 55-65° C. the solution is stirred for at least 1 hour
b6) the precipitate is isolated by filtration and the filter cake is washed with isopropyl acetate
b7) the product is dried to yield Intermediate XVIII.

29. The method of manufacturing Intermediate XVIII according to claim 28, wherein in step b5) after cooling to 60° C. seeding crystals of Intermediate XVIII are added and the solution is then stirred for at least 1 hour.

30. The method of manufacturing Intermediate XVIII according to claim 21, wherein step d) involves a recrystallization of Intermediate XVII from tetrahydrofuran to yield Intermediate XVIII.

31. The method of manufacturing Intermediate XVIII according to claim 30, wherein the recrystallization step d) of Intermediate XVII from tetrahydrofuran to yield Intermediate XVIII involves the following steps:
c1) Intermediate XVII is suspended in 7.0 volume parts (VP) tetrahydrofuran
c2) active carbon is added and the mixture is heated to reflux and stirred for at least 30 minutes
c3) the solution is filtered and concentrated to 2.5 volume parts
c4) the concentrated solution is heated to 50 to 60° C. and then stirred for at least 30 minutes
c5) 2.5 VP isopropyl acetate is added and the solution is stirred for at least 30 minutes
c6) the mixture is cooled to 7 to 13° C.
c7) the precipitate is isolated and then washed with acetonitrile
c8) the precipitate is dried in vacuo to yield Intermediate XVIII.

32. The method of manufacturing Intermediate XVIII according to claim 31, wherein in step c4) the concentrated solution is heated to 55° C. and then seeding crystals of Intermediate XVIII are added and the solution is then stirred for at least 30 minutes.

33. The method of manufacturing Intermediate XVIII according to claim 31, and wherein in step c2) 10 m % active carbon is added, wherein in step c6) the mixture is cooled to 10° C. and wherein in step c7) the precipitate is isolated by filtration and the filter cake is then washed with acetonitrile.

34. Method of recrystallizing Intermediate XIX 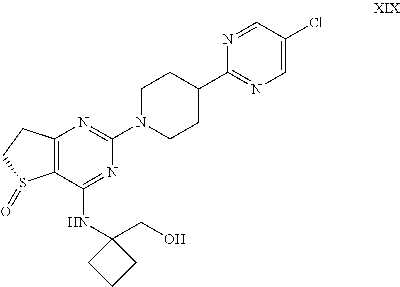 [SMILES: O=[S@@]1CCc2nc(N3CCC(c4ncc(Cl)cn4)CC3)nc(NC3(CO)CCC3)c21]
from a mixture of n-propanol and water to yield the PDE4B-inhibitor of formula XX in its crystalline form B 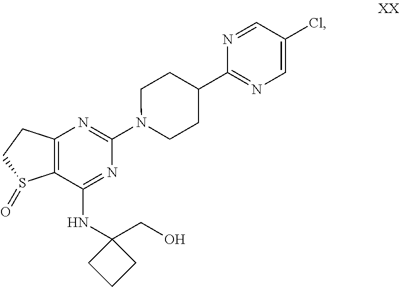 [SMILES: O=[S@@]1CCc2nc(N3CCC(c4ncc(Cl)cn4)CC3)nc(NC3(CO)CCC3)c21]
having a powder x-ray diffraction pattern comprising peaks at the following 2 Theta values measured using CuKα radiation: 19.18±0.2; 21.30±0.2; 24.28±0.2; 23.82±0.2 and 4.78±0.2,
and not comprising a peak at the following 2 Theta value measured using CuKα radiation: 8.76±0.2,
wherein this method involves the following steps:
d1) Intermediate XIX is dissolved in a mixture of 5.0 VP of n-propanol and of 1.25 VP of water under reflux conditions
d2) the solution is kept at 75 to 85° C. and filtered
d3) the solution is washed with 2.0 VP of n-propanol
d4) the solution is again heated to reflux and afterwards cooled to 65-75° C.
d5) seeding crystals of Compound XX are added and the mixture is stirred for at least 1 hour
d6) the mixture is then cooled to 15 to 25° C. within at least 90 minutes
d7) after stirring for at least 30 minutes the mixture is then heated again to a temperature between 35-45° C. within at least 30 minutes to yield crystalline form B of Compound XX latest within 4 hours
d8) the mixture is cooled to room temperature and 10.0 VP of n-propanol is added
d9) the mixture is cooled to 0-5° C. and after at least 1 hour the PDE4B-inhibitor of formula XX in crystalline form B is isolated
d10) after washing with n-propanol the PDE4B-inhibitor of formula XX in crystalline form B is dried.

35. The method of recrystallizing Intermediate XIX from a mixture of n-propanol and water to yield the PDE4B-inhibitor of formula XX in its crystalline form B according to claim 34, wherein in step d7) after stirring for at least 30 minutes the mixture is then heated again to a temperature of 40° C. within at least 30 minutes to yield crystalline form B of Compound XX latest within 4 hours.

36. The method of recrystallizing Intermediate XIX from a mixture of n-propanol and water to yield the PDE4B-inhibitor of formula XX in its crystalline form B according to claim 34, wherein in step d2) the solution is kept at 80° C., wherein in step d4) the solution is again heated to reflux and afterwards cooled to 70° C. and wherein in step d6) the mixture is cooled to 20° C. within at least 90 minutes.

37. A method of manufacturing Intermediate XVII 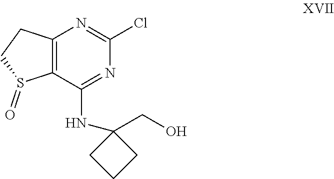 [SMILES: O=[S@@]1CCc2nc(Cl)nc(NC3(CO)CCC3)c21]
by oxidizing the Intermediate XVI 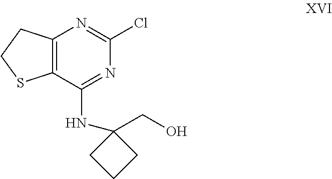 [SMILES: OCC1(Nc2nc(Cl)nc3c2SCC3)CCC1]
in the presence of S-(−)-Binaphthol, Ti(OiPr)₄ and t-BuOOH with a reduced amount of the catalyst Ti(OiPr)₄,
wherein this method involves the following steps e1) to e5)
e1) suspension of S-(−)-binaphthol in dichloromethane under inert atmosphere,
e2) addition of Ti(OiPr)₄ to the suspension and preincubation of this mixture for at least 1 hour,
e3) after that preincubation an amount of Intermediate XVI is added to the mixture that is ≥90-fold in excess to the amount of the catalyst Ti(OiPr)₄ and the suspension is incubated for at least one hour,
e4) then t-BuOOH is added and the mixture is stirred until reaction completion
e5) the precipitate containing Intermediate XVII is isolated.

38. The method of manufacturing Intermediate XVII according to claim 37, wherein in step e4) the t-BuOOH is added portion wise and the mixture is stirred until reaction completion.
